(19) United States
(12) Patent Application Publication
(10) Pub. No.: US 2025/0144247 A1
(43) Pub. Date: May 8, 2025

(54) FORMULATIONS TO ENHANCE THE DELIVERY OF GENE THERAPY VECTORS

(71) Applicant: University of Iowa Research Foundation, Iowa City, IA (US)

(72) Inventors: Ashley Cooney, Iowa City, IA (US); Patrick Sinn, Iowa City, IA (US); Paul B. McCray, JR., Iowa City, IA (US)

(21) Appl. No.: 18/936,764

(22) Filed: Nov. 4, 2024

Related U.S. Application Data
(60) Provisional application No. 63/595,608, filed on Nov. 2, 2023.

(51) Int. Cl. A61K 48/00; C12N 15/86

(52) U.S. Cl. CPC A61K 48/0058; A61K 48/0033; A61K 48/0075; C12N 15/86; C12N 2750/14143

(57) ABSTRACT

Method to enhance the delivery of one or more gene therapy vectors to cells comprising contacting said cells with a hypertonic solution before. after or simultaneously with said one or more gene therapy vectors.

### PRIORITY

This application claims the benefit of priority of U.S. Provisional Patent Application No. 63/595,608, filed on Nov. 2, 2023, which is incorporated by reference herein in its entirety.

### GOVERNMENT FUNDING

This invention was made with government support under HL152960 and HL133089 awarded by the National Institutes of Health. The government has certain rights in the invention.

### BACKGROUND

Gene therapy for lung diseases (such as cystic fibrosis, surfactant deficiencies, primary ciliary dyskinesia, others) is limited by the ability to efficiently deliver therapeutic cargoes to target airway or alveolar epithelial cells. The most common agents used in laboratory in vitro and in vivo studies to enhance gene transfer vector transduction are hypotonic solutions, EGTA or other calcium chelators, lysophosphatidylcholine (LPC), or chemical or mechanical injury. None of these methods are FDA approved, and some may be toxic or injurious to lung tissue.

### SUMMARY

Provided herein are novel compositions and methods that when used in conjunction with a gene transfer vector (e.g., adenovirus, adeno-associated virus, or lentivirus) markedly enhances the gene transfer efficiency.

One aspect provides a method to enhance the delivery of one or more gene therapy vectors to cells comprising contacting said cells with a hypertonic solution before, after or simultaneously with said one or more gene therapy vectors or any gene therapy delivery vehicle so as to enhance and/or provide improved enhanced gene delivery/transduction as compared to a method in which a hypertonic solution is not present/used. One aspect provides a method to enhance the delivery of one or more gene therapy vectors to a subject in need thereof comprising administering to said subject a hypertonic solution before, after or simultaneously with said one or more gene therapy vectors. Another aspect provides a method to restore cystic fibrosis transmembrane conductance regulator (CFTR) function to cells of subjects with cystic fibrosis comprising contacting said cells with a hypertonic solution before, after or simultaneously with said one or more gene therapy vectors/any gene addition or gene editing approaches for, for example, such as genetic diseases.

In one aspect, the cells comprise epithelial cells, such as pulmonary epithelial cells, or other cell lung types, cartilage and/or neuronal cells. In one aspect, the subject has a disease or disorder of a hollow organ, such as the respiratory tract, sinus, lung, intestinal tract, mouth, esophagus, stomach, bladder, gallbladder, reproductive tract or heart. In one aspect, the disease or disorder is a genetic disease or disorder. In one aspect, the disease or disorder comprises cystic fibrosis, primary ciliary dyskinesia (PCD), surfactant deficiencies, underlying targets of interstitial lung disease, atopic lung disease, asthma, bronchiectasis, bronchiolitis obliterans, congenital hypoventilation, cystic lung diseases, Alpha-1 antitrypsin (AAT) deficiency, alveolar diseases in alveolar type 2 cells, lung cancer, Crohn's, ulcerative colitis, colon cancer, Lynch syndrome, familial adenomatous polyposis (FAP), inflammatory bowel disease, oral cancer, achalasia, GERD, esophageal cancer, Barrett's syndrome, stomach cancer, bladder cancer, bladder exstrophy, incontinence, Giltelman syndrome, gallstones, cardiomyopathies, channelopathies, heart disease, inherited heart conditions, hypertrophic cardiomyopathy (HCM), idiopathic or familial dilated cardiomyopathy (DCM), arrhythmogenic right ventricular cardiomyopathy (ARVC), restrictive cardiomyopathy, Carney complex or cancer, including, but not limited to, a sarcoma (such as osteosarcoma and Kaposi's sarcoma), carcinoma, lung cancer, adenocarcinoma, adenocarcinoma of the lung, squamous carcinoma, squamous carcinoma of the lung, malignant mixed mullerian tumor, head and/or neck cancer, breast cancer, esophageal cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, stomach cancer, prostate cancer, testicular cancer, ovarian cancer, cervical cancer, endometrial cancer, uterine cancer, pancreatic cancer, colon cancer, rectal cancer, colorectal, gastric cancer, kidney cancer, bladder cancer, spleen cancer, thymus cancer, thyroid cancer, brain cancer, neuronal cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, neuroblastoma, hereditary non-polyposis cancer (HNPC), and/or colitis-associated cancer.

In one aspect, the gene therapy vector is a viral vector, such as adenovirus, adeno-associated virus (AAV), lentivirus, retrovirus, bocavirus, oncolytic vectors and/or virus like particles (VLPs). In one aspect, the gene therapy vector is a plasmid, mRNA, peptide, protein, exosomes, microvesicles, viral nucleic acid, phage nucleic acid, phage, cosmid, artificial chromosome, or via transfer of genetic material in cells or carriers such as cationic liposomes, lipid nanoparticles, polymeric nanoparticles or other biomaterials.

In one aspect, the hypertonic solution is a hypertonic salt solution. In one aspect, the hypertonic salt solution comprises one or more monovalent salts, including sodium chloride (NaCl), potassium chloride (KCl), and/or lithium chloride (LiCl). In one aspect, the hypertonic salt solution comprises one or more divalent salts, including calcium chloride (CaCl₂), magnesium sulfate (MgSO₄), magnesium chloride (MgCl₂), zinc cholirde (ZnCl₂), zinc sulfate (ZnSO₄), calcium lactate (C₆H₁₀CaO₆), ferrous sulfate (FeSO₄), calcium glycerylphosphate and/or ferrous chloride (FeCl₂). In one aspect, the hypertonic solution is a charged sugar or any solution demonstrating increased ionic strength, including N-methyl-D-gluconate.

In one aspect, the salt concentration is from about 1% to about 8%, including from about 3.5% to about 7% and from about 3.6-7.2%.

In one aspect the hypertonic solution is administered by inhalation, instillation, topical delivery or injection, including intra-tumoral injection.

### BRIEF DESCRIPTION OF THE DRAWINGS

FIGS. 1A-1E. Increasing saline tonicity enhances apical Ad gene transfer in airway epithelial cells. (A) Ad-CMV-eGFP (MOI=250) was co-delivered to HAE with DMEM or NaCl ranging from 1-7% (final concentration) and imaged 5 days post-transduction. 0.33 cm² transwells were imaged at 2×. (B) Percentage of GFP+ cells in each condition quantified by flow cytometry. (C) Percentages of GFP+ cells by cell type from 1-7% NaCl. (D) Tight junction permeability was measured by the passage of dextran from the apical to basolateral media. HAE were left untreated or treated with NaCl (1-7%) for 2 h. Following the treatment, apical dextran was applied, and basolateral media was collected 30 min later and fluorescent units were measured using a plate reader. (E) Correlative % GFP (R²=0.75) from FIG. 1B and Dextran Permeability (R²=0.86) from FIG. 1D were plotted together. N=3, *P<0.05, **P<0.005, ***P<0.0005, ****P<0.00005. Statistical differences were determined by one-way ANOVA.

FIGS. 2A-2F. Apically applied Ad-CFTR+3.6% NaCl restores anion transport defect in CF airway epithelial cells. (A, B) Short circuit current measurements of CF HAE alone or treated with Ad-CFTR or Ad-CFTR+3.6% NaCl (MOI=250) compared to non-CF. Responses to Forskolin and IBMX (F&I) and GlyH-101 (GlyH) are reported. (C, D) Short circuit conductance in response to F&I and GlyH (N=5). (E) Representative images of parallel CF cultures transduced with Ad-GFP (MOI=250) and (F) quantified by flow cytometry (N=3). Inset: representative western blot of CF HAE untreated or transduced with Ad-CFTR or Ad-CFTR+3.6% NaCl. Western blot was probed for CFTR (green) and Vinculin (red) (loading control). *P<0.05, **P<0.005, ***P<0.0005. Statistical differences were determined by Student's t-test or one-way ANOVA.

FIGS. 3A-3G. Entry is dependent on ionic strength but not osmolarity, osmolytes, or pH. (A) Isotonic NaCl (0.9%), Isotonic NaCl+the non-ionic osmolyte mannitol with equivalent osmolarity (620 mM, 1 230 mOsmol/1) to 3.6% NaCl, NaGluconate (3.6%), and KCl (3.6%) were co-delivered apically to HAE with Ad-GFP (MOI=250) for 2 h. 5 days post-transduction, cells were imaged and (B) GFP expression was quantificd by flow cytometry. (C) Ad-GFP was co-delivered with NaCl (3.6%) at pH 5, 6, 7, 7.4 and 8 f or 2 h. 5-day post-transduction GFP was quantified by flow cytometry. (D) KCl, mannitol, or N-methyl-D-glucamine (NMDG) gluconate 1-7% was formulated with Ad-GFP (MOI=250) and applied to the apical surface of HAE for 2 h. Cultures were imaged 5 days post-transduction and GFP expression was quantified by flow cytometry for (E) KCl, (F) mannitol, or (G) NMDG gluconate. N=3, *P<0.05, **P<0.005, ****P<0.00005. Statistical differences were determined by one-way ANOVA.

FIGS. 4A-4C. NaCl-mediated transduction requires receptor and low pH entry step. Ad5 utilizes CXADR for entry and Ad21 utilizes CD46. (A) CRISPR/Cas9 with gRNA targeted to CXADR was electroporated into HAE and allowed to re-differentiate at air liquid interface for 3 weeks. In parallel with non-electroporated cells from the same donor, Ad5-GFP and Ad21-GFP were applied apically to HAE with or without NaCl for 2 h. Cultures were imaged 5 days post-transduction and representative images are shown. (B) GFP was quantified by flow cytometry. Inset: Western blot for CXADR in parallel on CXADR^{KO} or scrambled airway epithelial cultures. (C) HAE were pre-treated with endosomal acidification inhibitors chloroquine (CQ) (200 μM) or bafilomycin A1 (1 μM) for 2 h. Next, Ad5-GFP (MOI =250) was delivered with or without NaCl (3.6%) for 2 h. GFP expression was quantified by flow 5 days post-transduction. N=3, *P<0.05, **P<0.005, ****P<0.00005. Statistical differences were determined by one-way ANOVA.

FIGS. 5A-5G. Increasing saline tonicity enhances GP64 pseudotyped lentiviral and AAV transduction. (A) GP64 pseudotyped HIV-CMV-cGFP (MOI=50) was co-delivered to HAE with DMEM, 3.5%, 4%, or 4.5% NaCl for 2 h and then removed. 1-week post-transduction GFP expression was quantified by flow cytometry. (B) Parallel CF cultures were transduced with GP64 HIV-CMV-eGFP. GFP expression was visualized by fluorescence microscopy and quantified by flow cytometry 5 days later. (C, D) As described in FIG. 2, CF HAE were transduced with GP64-HIV-CFTR (MOI=50) formulated with DMEM or 4.5% NaCl for 2 h and incubated for 1 week. Short circuit current of F&I and GlyH in untreated or treated CF cells compared to non-CF. (E) AAV with the 2.5T capsid expressing CMV-eGFP was co-delivered (MOI=10 0 0 0 0) with DMEM or 4.5% NaCl for 2 h. HAE were pre-treated with doxorubicin (1 μM) for 24 h prior to transduction. 2 weeks post-transduction, airway cultures were imaged and GFP was quantified by flow cytometry. (F, G) CF airway cultures were treated overnight with doxorubicin (1 μM) and the next day cells were treated with AAV2.5T-CFTR alone or with 4.5% NaCl for 2 h. Two weeks post-transduction, electrical properties were measured by Ussing chambers as described in (C, D). N=3, *P<0.05, **P<0.005. Statistical differences were determined by Student's t-test or one-way ANOVA.

FIGS. 6A-6L. Increased saline tonicity enhances transduction of mouse and pig airways. (A) 6-8-week-old Balb/c mice received intratracheal Ad-luciferase formulated with NaCl (0.9% or 7% final concentration). Mice were imaged by IVIS 5 days after delivery (N=5). (B) Representative images of luciferase expression in mouse nose and lungs quantified in (A). (C-L) Ad-GFP was formulated 1:1 with 1.8% (0.9% final concentration) or 10% NaCl (5% final concentration) and acrosolized intratracheally to newborn pigs. 5 days after delivery, lungs were divided into 20 regions as previously reported (2). (C) Combined GFP+scores (0=no expression, 1=low expression, 2=moderate expression, and 3=high expression). (D, E) Lungs were fixed, processed, sectioned, and counterstained with DAPI. Representative 10× and 20× images of small and large airways from 5% NaCl condition shown. (F) GFP+cells quantified in small (<500 μm²) and large (>500 μm²) in pigs that received Ad-GFP formulated with 0.9% or 5% NaCl. (G-L) Representative images of cell types transduced in 5% NaCl condition. (G) Transduced ciliated cells detected by immunofluorescence using acetylated α-tubulin. (H-L) Cytokeratin 5 (CK5) staining representing basal cells transduced in large airways (H) and small airways (I). (J-L) Representative images of submucosal gland transduction; empty arrow pointing to transduced ductal cells. Arrows indicate Ad-GFP expression in counterstained α-tubulin or CK5 cells. Pig experiment: n=1 animal per condition. 10 slides per lung were evaluated. *P<0.05. ****P<0.0 0 0 05. Statistical differences w ere determined by Student's t-test.

### DESCRIPTION

A fundamental challenge for cystic fibrosis (CF) gene therapy is ensuring the transduction of sufficient airway epithelial cells to achieve therapeutic correction. FDA approved reagents were screened for their ability to enhance viral vector of transduction of primary human airway epithelial cells. Hypertonic saline was identified as a candidate that conferred a clear benefit. Hypertonic saline (3-7%) is frequently administered as an agent to enhance mucociliary clearance in people with CF. Hypertonic saline use transiently disrupts epithelial cell tight junctions, but its ability to improve gene transfer has not been investigated. Here it was asked if increasing NaCl tonicity in the vector formulation enhances the transduction efficiency of gene therapy vectors, including adenovirus, AAV, and lentiviral vectors. It was observed that vectors formulated in vitro with 3-5% NaCl exhibited markedly increased transduction, leading to phenotypic correction of the anion channel defect in primary cultures of CF epithelial cells. It was also found that the NaCl-enhanced transduction is not dependent on ionic osmolytes, osmolarity, or pH; however, entry remains receptor-dependent and low pH endosomal release. Gene transfer in vivo was also enhanced in mouse and pig airways by formulating vectors in NaCl (5% or 7%). These findings have broad implications for gene therapy for cystic fibrosis and other genetic lung diseases, as well as other diseases.

Thus, provided herein are novel compositions and methods that when used in conjunction with a gene transfer vector (e.g., adenovirus, adeno-associated virus, or lentivirus) markedly enhances the gene transfer efficiency.

### Definitions

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any clement described herein, and/or the recitation of claim elements or use of “negative” limitations. “Plurality” means at least two.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.” As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The terms “patient,” “individual,” or “subject” are used interchangeably herein, and refer to a mammal, particularly, a human. The patient may have mild, intermediate or severe disease. The patient may be an individual, at risk of developing a disease, in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters, and primates. Mammals include, but are not limited to, humans, farm animals, sport animals and pets.

As used herein, “health care provider” includes either an individual or an institution that provides preventive, curative, promotional or rehabilitative health care services to a subject, such as a patient. In one embodiment, the data is provided to a health care provider so that they may use it in their diagnosis/treatment of the patient.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating,” “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

### Hypertonic Solutions

Provided herein are hypertonic solutions. A hypertonic solution is any external solution that has a high solute concentration and low water concentration compared to body fluids. Hypertonic salt solutions (ionic osmolyte) include mono-or di-valent salt solutions or any charged molecule with ionic strength. Hypertonic monovalent salt solutions include, but are not limited to, sodium chloride (NaCl), potassium chloride (KCl), and/or lithium chloride (LiCl). Hypertonic divalent salt solutions include, but are not limited to, calcium chloride (CaCl₂), magnesium sulfate (MgSO₄), magnesium chloride (MgCl₂), zinc cholirde (ZnCl₂), zinc sulfate (ZnSO₄), calcium lactate (C₆H₁₀CaO₆), ferrous sulfate (FeSO₄), calcium glycerylphosphate and/or ferrous chloride (FeCl₂).

The concentration of the salt solutions can range from about 1% to 8%, such as 1%, 1.5%, 2%, 2.5%, 3%, 3.5%, 4%, 4.5%, 5%, 5.5%, 6%, 6.5%, 7%, 7.5% or 8%, including greater than 1%, up to about 7%, including 3.5 to 7%, and 3.6-7.2%.

### Gene Transfer Vectors

Define “gene therapy vectors” are vehicles designed to deliver therapeutic genetic material, such as a working gene, directly into a cell. Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. The gene transfer vector can comprise DNA, RNA (e.g., antisense nucleic acid molecule, a small interfering RNA (siRNA), microRNA (miRNA) a short hairpin RNA (shRNA), synthetic mRNA and/or coding sequence), peptide, protein, and/or viral associated vectors, including but not limited to, adenovirus, adeno-associated virus (AAV), lentivirus, retrovirus, bocavirus, oncolytic vectors and/or virus like particles (VLPs). The gene therapy vector can also comprise exosomes, microvesicles, or nanoparticles (e.g., spherical, polymeric particles composed of natural or artificial polymers; can range in size between 10 and 500 nm), including lipid nanoparticles (e.g., nucleic acid-based cargo vehicles) and/or polymeric nanoparticles.

### Diseases/Disorders

Provided herein is a method to improve gene therapy vector transduction, such as at epithelial surfaces, including the respiratory tract (e.g., pulmonary airway epithelial cells, and alveolar epithelial cells), gastrointestinal (GI) tract, and/or reproductive tract.

For example, the methods and compositions provided herein can treat lung disease, such as any genetic lung disease, including, but not limited to, cystic fibrosis, primary ciliary dyskinesia (PCD), surfactant deficiencies, underlying causes of interstitial lung disease, atopic lung disease, asthma, bronchiectasis, bronchiolitis obliterans, cystic lung diseases, Alpha-1 antitrypsin (AAT) deficiency, alveolar diseases in alveolar type 1 and/or type 2 cells, and/or lung cancer.

The methods and compositions provided herein can treat disease of hollow organs, such as genetic disease of hollow organs, including, but not limited to, visceral organs that are hollow tubes or pouches, such as intestine (e.g., small, large and colon; e.g., Crohns' and ulcerative colitis, cancer, such colon cancer; colon cancer, Lynch syndrome, familial adenomatous polyposis (FAP), inflammatory bowel disease); mouth (e.g., oral cancer); esophagus (e.g., achalasia, GERD, esophageal cancer, Barrett's syndrome); stomach (e.g., stomach cancer); bladder (e.g., bladder cancer, bladder exstrophy, incontinence, Giltelman syndrome); gallbladder (e.g., gallstones), or that include a cavity, such as the heart (e.g., cardiomyopathies, channelopathies, heart disease, inherited heart conditions (hypertrophic cardiomyopathy (HCM), idiopathic or familial dilated cardiomyopathy (DCM), arrhythmogenic right ventricular cardiomyopathy (ARVC), restrictive cardiomyopathy), Carney complex), pancreatic diseases, kidney diseases, vascular diseases, bone diseases, spleen diseases, car diseases, skin diseases, and/or blood diseases.

In one aspect, the hypertonic solution can be used to enhance gene therapy vector transduction in cancer, such as by intra-tumoral injection. Cancer includes, but is not limited to, sarcomas (such as osteosarcoma and Kaposi's sarcoma) and carcinomas. The cancer can include, but is not limited to, lung cancer, adenocarcinoma, adenocarcinoma of the lung, squamous carcinoma, squamous carcinoma of the lung, malignant mixed mullerian tumor, head and/or neck cancer, breast cancer, esophageal cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, stomach cancer, prostate cancer, testicular cancer, ovarian cancer, cervical cancer, endometrial cancer, uterine cancer, pancreatic cancer, colon cancer, rectal cancer, colorectal, gastric cancer, kidney cancer, bladder cancer, spleen cancer, thymus cancer, thyroid cancer, brain cancer, neuronal cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, neuroblastoma, hereditary non-polyposis cancer (HNPC), and/or colitis-associated cancer.

### Administer

Administration of the hypertonic solution and the genetic material in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the genetic material and compositions of the invention may be essentially continuous over a preselected period of time or may be in a series of spaced doses. Both local and systemic administration is contemplated.

The hypertonic solution can be administered simultaneously with the genetic material, before or after administration of the genetic material, or with the genetic material mixed together with the hypertonic solution.

It will be appreciated that the amount of genetic material and hypertonic solution for use in treatment will vary not only with the particular treatment selected but also with the route of administration, the nature of the condition being treated and the age and condition of the patient. Ultimately the attendant health care provider can determine proper dosage. In addition, a pharmaceutical composition can be formulated as a single unit dosage form.

Thus, one or more suitable unit dosage forms comprising the genetic material and/or the hypertonic solution can be administered by a variety of routes including parenteral (including subcutaneous, intravenous, intramuscular and intraperitoneal), oral, rectal, dermal, transdermal, intrathoracic, intrapulmonary and intranasal (respiratory, including inhaled) routes. The formulations may, where appropriate, be conveniently presented in discrete unit dosage forms and may be prepared by any of the methods well known to the pharmaceutical arts.

Some aspects provide oral dosage forms, inhaled/aerosolized forms (e.g., PulmoSal, HyperSal; such aerosol hypotonic solution and vector administration together). Alternatively, any hollow organ can be filled with the hypertonic solutions provided herein, as well as intra-tumoral injection (with optional cancer treating agents, such as chemotherapy or immunotherapy).

The present description is further illustrated by the following examples, which should not be construed as limiting in any way. The contents of all cited references (including literature references, issued patents, published patent applications as cited throughout this application) are hereby expressly incorporated by reference.

### Example

### High Ionic Strength Vector Formulations Enhance Gene Transfer to Epithelia.

A fundamental challenge for cystic fibrosis (CF) gene therapy is ensuring sufficient transduction of airway epithelia to achieve therapeutic correction. Hypertonic saline (HTS) is frequently administered to people with CF to enhance mucus clearance. HTS transiently disrupts epithelial cell tight junctions, but its ability to improve gene transfer has not been investigated. Here, it demonstrated that increasing the concentration of NaCl enhances the transduction efficiency of gene therapy vectors, such as adenovirus, AAV, and lentiviral vectors. Vectors formulated with 3-7% NaCl exhibited markedly increased transduction f or all three platforms, leading to anion channel correction in primary cultures of human CF epithelial cells and enhanced gene transfer in mouse and pig airways in vivo. The mechanism of transduction enhancement involved tonicity but not osmolarity or pH. Formulating vectors with a high ionic strength solution is a simple strategy to greatly enhance efficacy and immediately improve preclinical and/or clinical applications.

### Introduction

Cystic fibrosis (CF) is an autosomal recessive genetic disease caused by pathogenic variants of the cystic fibrosis transmembrane conductance regulator (CFTR) gene. CFTR protein conducts Cl⁻ and HCO₃⁻ anions at the apical surface of epithelial cells. Decreased activity or loss of CFTR in the airways leads to a series of airway complications including bacterial colonization, chronic inflammation, and mucus accumulation. Numerous studies have established that CFTR complementation restores anion transport both in vitro and in vivo (1-3). The advent of the small molecule therapy of celexacaftor, tezacaftor, and ivacaftor (ETI), trade name Trikafta, demonstrated that restoring CFTR improves lung function and quality of life in people with CF. Although ETI treatment offers benefits the majority of people with CF, there remains a pressing need to develop treatments for all people with CF. Gene therapy by CFTR complementation can restore anion channel activity regardless of the mutation. Despite numerous clinical trials (reviewed in (4)), no CF gene therapies have advanced past Phase II trials due to low efficacy of CFTR gene transfer.

The conducting airways are challenging to tranduce using topically applied gene therapy reagents due to an enormous surface area and multiple host defense mechanisms to resist viral vector uptake (4). Vehicle formulations such as lysophosphatidylcholine (LPC) (2, 5), EGTA (6, 7), methylcellulose (8), and perflurocarbon (9) improve viral vector transduction and subsequent transgene expression in cultured cells and animal models; however, none of these materials are currently FDA approved for pulmonary administration. An ideal vector formulation that enhances gene transfer would be FDA approved and cost. Hypertonic saline (HTS) is FDA approved, Generally Recognized As Sage (GRAS), and offers a translational advantage over other vector formulations not currently approved. HTS is commonly acrosolized at 3-7% as a topical lung treatment for people with CF to improve mucociliary transport. HTS transiently disrupts tight junctions and exhibits multiple actions including: a mucolytic agent (disrupting mucus gel), an expectorant (hydrolyzing airway surface liquid), a mucokinetic agent (promoting cough-mediated clearance) and increasing ion flux of two thiols (glutathione and thiocyanate) that protect against oxidative injury (10). Thus, HTS hydrates surface epithelia, breaks ionic bonds, dislodges mucus, and enhances mucociliary clearance (11). Clinical benefits and safety track records of this treatment are well established for people with CF.

Herein, reagents approved for topical airway delivery were screened for their ability to decrease transepithelial resistance to increase access to basolateral receptors and enhance gene transfer. Adenoviral (Ad) vectors, adeno-associated viral (AAV) vectors, or lentiviral (LV) vector human immunodeficiency virus (HIV) were formulated with 1-7% NaCl and the mixtures were applied to the apical surface of cultured primary human airway epithelial cells (HAE). It was found that an increase in NaCl tonicity of the vector formulation correlated with enhanced gene transfer in a dose dependent fashion and functionally restored CFTR anion transport in vitro. Focused entry studies with Ad suggest vector entry remains receptor-mediated, improves with increasing ionic strength and requires a low pH endosomal step. Finally, it was asked if vectors delivered in increased NaCl tonicity solutions enhanced gene transfer ex vivo and in vivo. Ad and AAV transduction was significantly increased in pig explants ex vivo. Ad5 was delivered with NaCl to both mouse (7%) and pig airways (5%) and observed significantly enhanced gene transfer was observed. Additionally, GP64 pseudotyped HIV lentivirus was aerosolized with NaCl (5%) to pig airways and widespread reporter gene expression greater than the isotonic saline control was observed. Together, these findings indicate that formulating gene delivery vectors, such as viral vectors, with hypertonic solutions, such as hypertonic saline, improves transduction.

### Materials and Methods

### Study Design

This study was done to validate that increasing saline tonicity enhanced viral transduction in a dose dependent manner across Ad, AAV and lentiviral vector platforms. All in vitro experiments were done by applying a viral vector alone or formulated with NaCl for 2 hours. To determine the mechanism of entry, osmolarity, ionic strength, pH, endocytosis inhibitors, micropinocytosis inhibitors, and inhibitors of a low pH endosome were compared. Two independent receptor-dependent approaches were also performed. In vivo studies in mice and pigs confirmed in vitro findings by increased transgene expression in the airways when vectors were formulated with NaCl (5% or 7%).

### Human Airway Epithelial Cells

The University of Iowa In Vitro Models and Cell Culture Core cultured and maintained human airway epithelial cells (HAE) as previously described (33). Briefly, following enzymatic disassociation of trachea and bronchus epithelia, the cells were seeded onto collagen-coated, polycarbonate transwell inserts (0.4 μm pore size; surface area=0.33 cm²; Corning Costar, Cambridge, MA). HAE were submerged in Ultroser G (USG) medium for 24 hours (37° C. and 5% CO₂) at which point the apical media is removed to encourage polarization and differentiation at an air-liquid interface. Transepithelial electrical resistance was measured using an Ohmmeter (Ω·μm²)

### Viral Vector Production and Formulation

All vectors were produced by the University of Iowa Viral Vector Core (https://medicine.uiowa.edu/vectorcore/). Ad5 and Ad21 CMV-cGFP CMV-mCherry, or F5Tg83-CFTR was produce, purified, and titered as previously described (34). AAV2/2.5T, AAV2/H22 CMV-eGFP or or F5Tg83-CFTRAR was produced by triple transfection. Lentiviral HIV-CMV-cGFP or PGK-CFTR vectors pseudotyped with GP64 were produced by a four-plasmid transfection method as previously described (35, 36) and titered using droplet digital PCR (37) and/or by flow cytometry. Similarly, VSVG, JSRV and BaEV pseudo-typed lentiviral vectors were made by four-plasmid transfection and titered by flow cytometry. Viral vectors were formulated with indicated NaCl concentrations (presented as final concentrations). The following MOIs were used for each vector: Ad=250, AAV=100 000, and HIV=50. Ad studies were performed by mixing pharmaceutical grade 7.2% NaCl with Ad-GFP (3.6% final). Additional concentrations were made from a 10% NaCl solution of Sodium Chloride (Research Products International, Mount Prospect, IL) in UltraPure Distilled Water (Invitrogen, Waltham, MA). Vectors were formulated with NaCl in a final volume of 50 μl and applied to the apical surface of HAE for 2 h. AAV transduced cultures were pre-treated with indicated concentrations of doxorubicin (1 μM) for 24 h prior to 2 h vector application. Other formulations tested include lysophosphatidylcholine (LPC) (9008-30-4, Millipore Sigma, St. Louis, MO), Ethylene glycol-bis-2-aminocthylether)-N,N,N′N′-tetraacetic acid (EGTA) (Research Products International, Mount Prospect, IL), and surfactant (Infasurf, pharmaceutical grade).

### Fluorescence Microscopy and Flow Cytometry

GFP images were acquired using a Keyence All-in-one Fluorescence Microscope BZ-X series (Osaka, Japan). 0.33 cm² Transwells were imaged at 2× magnification. GFP expression was quantified by flow cytometry as previously reported (36). Briefly, cells were stained with a fixable LIVE/DEAD stain (Thermo Fisher Scientific, Waltham, MA, USA), lifted in Accutase at 37° C. for 30 minutes, and run through an Attune NxT Flow Cytometer (Thermo Fisher Scientific, Waltham, MA, USA). Cells were treated with the Foxp3/Transcription Factor Staining Buffer Set (Thermo Fisher Scientific, Waltham, MA, USA) according to manufacturer's recommendations and stained for 1 hour at 37° C. with the following antibodies: NGFR (345110; 1:600, BioLegend, San Diego, CA, USA), α-tubulin (NB100-69AF405, 1:300, Novus, Centennial, CO, USA) and CD66c (12-0667-42, 1:600, Invitrogen, Waltham, MA). Expression was gated on live cells. Immunofluorescence staining of pig tissue sections was performed using acetylated α-tubulin (D20G3, 1:200, Cell Signaling Technology, Danvers, MA) or cytokeratin 5 (CK5) (Polyl9055, 1:500, San Diego, CA). Primary antibodies were detected using a goat anti-rabbit Alexa 546 secondary anti-body (A-11035, 1:600, Thermo Fisher Scientific, Waltham, MA).

### LDH Release Assay

LDH release was quantified according to the manufacturer's recommendations (LDH-Glo Cytotoxicity Assay, Promega, Madison, WI). Briefly, basolateral media from each condition was collected in a 96 well plate. The LDH detection reagents were mixed in a 1:1 ratio and applied to each well and incubated for 30 minutes. Luminescence was recorded using a SpectraMax i3x plate reader.

### Dextran Permeability Assay

Tight junction permeability was assessed by passage of 3000-5000 average molecular weight Fluorescein isothiocyanate-dextran (FD4, Sigma-Aldrich, St. Louis, MO, USA) from the apical to basolateral side of well-differentiated human airway epithelia (HAE). Carbonyl cyanide 3-chlorophenylhydrazone (CCCP) (Sigma-Aldrich, St. Louis, MO, USA) serves as a positive control for membrane permeabilization and was pre-treated (50 μM) overnight prior to addition of dextran. 250 mM EGTA pre-treatment for 2 hours was used for a positive experimental tight junction disruption control. Cells were left untreated or pre-treated with NaCl ranging from 1-7% for 2 hours. At the time of the assay, pre-treatments were removed, and fresh basolateral media was added. 100 μl of 1 mg/ml dextran was applied apically for 30 minutes and 37° C. Basolateral media was assessed for fluorescent units using a SpectraMax i3x plate reader.

### SEM

Scanning electron microscopy (SEM) samples were processed by the Johns Hopkins Institute for Biomedical Sciences Microscope Facility as a fec for service (https://microscopy.jhmi.edu/Services/EMCostStruct.html).

### Electrophysiology

Short circuit current (I_{sc}) and conductance (G_{sc}) was measured as previously described (36). Briefly, cells were pre-stimulated with forskolin and IBMX (F&I) overnight and their bioelectric properties were quantified by Ussing chamber analysis. Assay protocol is as follows: amiloride, 4,4′-diisothiocyano-2,2′-stilbenedisulfonic acid (DIDS), F&I, and GlyH-101 (GlyH). Results are reported as change in short circuit current (ΔI_{sc}) and conductance (ΔG_{sc}) in response to F&I or GlyH.

### Western Blot

Protein from human airway epithelia (HAE) transduced with Ad-CFTR alone or Ad-CFTR+NaCl (3.6%) was harvested using RIPA buffer. 20 μg of each sample was loaded on a criterion Tris-Glycine 4-20% gel and run at 110 V for 1 hour and transferred overnight onto a PVDF membrane at 110 μA at 4° C. After membrane blocking, each membrane was probed using the following primary antibodies: mouse anti-CFTR 596 (1:1000) (UNC) or CAR anti-rabbit (CXADR) (1:100) (PA5-31175, Invitrogen, Waltham, MA) was applied for 2 hours. Following TBST washes, the LiCor secondary antibody was used at 1:10,000 for 30 minutes. The membrane was imaged using the Odyssey imager.

Sugar Tonicity and Ionic vs. Non-Ionic Osmolytes

Sodium chloride, mannitol, sodium gluconate, and N-methyl-d-glucamine (NMDG) gluconate were formulated 1-7% and co-delivered with Ad-GFP (MOI—250) for 2 h apically. Molarities and osmolarities are presented in Table 1. Sodium chloride (NaCl), mannitol, sodium gluconate, and potassium chloride (KCl) were made with equal percentages (3.6%) or equal molarity (1.23 mOsmol/l) to NaCl as shown in Table 2. Each solution was mixed with Ad-GFP (MOI =250) and applied apically to HAE for 2 h. GFP expression was quantified by flow cytometry 5 days post-transduction.

### CXADR Knockout (KO)

CRISPR/Cas9 was used to knockout the coxsackie and adenovirus receptor CXADR by nucleofection (Lonza, Basel Switzerland). Well-differentiated human airway epithelia (HAE) were lifted in TrypLE and electroporated with ribonucleoproteins (RNP). CXADR Exon 4 was targeted with the following guide RNAs: gRNA 1: ACGTAACATCTCGCACCTGAAGG (SEQ ID NO: 1) and gRNA 2: AGTACCTGCTAACCATGAAGTGG (SEQ ID NO: 2) (13). Alt-R S.p. HiFi Cas9 nuclease (1081058, IDT, Coralville, IA) was mixed with crRNA and trerRNA duplex to form the RNP. RNP and cells were mixed with the nucleofector solution and electroporated using program U-024 and seeded in a 6 well plate in Pneumacult ExPlus media. 2 days later, cells were lifted and reseeded on a Corning 3413 collagen coated membranes. Cells were maintained in Pneumacult ALI-M media for 3 weeks to differentiate. Ad5-GFP (CXADR-dependent) and Ad21-GFP (CXADR-independent) were formulated with DMEM or NaCl (3.6%) and applied to the apical surface of airway epithelia for 2 hours. GFP expression was quantified 5 days post-transduction by flow cytometry.

### Inhibitors of Endosomal Entry, Macropinocytosis, or Endosomal Acidification

Endosomal inhibitors Dynasore (400 μM) (D7693, Millipore Sigma, St. Louis, MO) and Methyl-β-CycloDextran (10 mM) (332615, Millipore Sigma, St. Louis, MO) were applied to well-differentiated human airway epithelia for 2 hours. Following the pre-treatment, cells were transduced with Ad-GFP alone or with Ad-GFP formulated with NaCl (3.6%) for 2 hours. GFP expression was quantified by flow cytometry 5 days later. Similarly, inhibitors of macropinocytosis, Amiloride (100 μM) (1019701, Millipore Sigma, St. Louis, MO) or 5-(N-Ethyl-N-isopropyl) amiloride (EIPA) (100 μM) (A3085, Millipore Sigma, St. Louis, MO) were applied to well-differentiated airway epithelia for 2 hours. Cells were next transduced with Ad5-GFP with or without NaCl (3.6%) for 2 hour and assayed for GFP expression 5 days later. Endosomal acidification inhibitors Choloroquine (CQ) (200 M) (C6628, Millipore Sigma, St. Louis, MO) and bafilomycin A1 (Baf A1) (1 μM) (19148, Millipore Sigma, St. Louis, MO) were used to pre-treat airway epithelia for 2 hours prior to Ad-GFP transduction in the presence or absence of NaCl (3.6%). GFP was quantified 5 days post-transduction.

### Measles (MeV) Infection of Human Airway Epithelia (HAE)

For apical infections of HAE, MeV in opti-MEM or opti-MEM+NaCl (4.5%) was placed on the apical surface of HAE. After infection, virus inoculums removed. For basolateral infections, cultures were inverted to expose the basolateral surface and MeV in opti-MEM or opti-MEM+NaCl (4.5%) was placed. After infection, basolateral-infected cultures were returned upright. All MeV infections took place for 4 hours in 37° C. at an MOI of 1 with a total inoculum of 50 μL.

### Pig Explants Transductions

0.25 cm² tracheal explants from newborn pigs were cultured on Surgifoam (1972, Ethicon, Raritan, NJ) for 3-5 days prior to transduction. Ad5-mcherry (1×10⁹ TU), AAVH22-GFP (2×10¹⁰ vg), and GP64 HIV-GFP (1.75×10⁷ TU) were applied with or without NaCl (5%) to the apical surface of the trachea by inverting the trachea onto a tissue culture dish containing the viral solution for 2 hours then returned to an upright position on Surgifoam. 5 days post-transduction, tracheal explants were mounted on a microscope slide and imaged for fluorescent expression using confocal microscopy. Expression was quantified using Image J (FIJI) by measuring fluorescence intensity per high power field.

### Adenoviral Transduction of Mouse Airways

6-8-week-old Balb/c mice were sedated with ketamine/xylazine (87.5+2.5 mg/kg). Ad-CMV-firefly luciferase was delivered intratracheally with 1×10⁹ TU formulated with NaCl (0.9% or 7% final) in a 50 ul volume. 5 days later mice were given intraperitoneal D-luciferin (50227, Millipore Sigma, St. Louis, MO) imaged for luminescence expression using the Xenogen IVIS-200.

### Viral Gene Transfer in Pig Airways

Newborn pigs were sedated using isoflurane and viral vector formulated with NaCl (0.9% or 5% final) and acrosolized 2 ml intratracheally using a MADgic Laryngo-Tracheal Mucosal Atomization Device (Teleflex, Morrisville, NC). Doses for each vector for each pig were as follows: 2.8×10¹⁰ infectious genomic units (IGU) of helper-dependent Ad-CMV-GFP and 7×10⁸ TU of GP64 HIV-CMV-GFP. Doses were prepared by mixing a 1.8% NaCl or 10% NaCl stock 1:1 with 1 ml of vector. 1 week later, pigs were humanely euthanized, and lungs were analyzed for GFP expression. Lungs were systematically divided and fixed in 4% paraformaldehyde, subjected to a sucrose gradient, and embedded in OCT for cryosectioning. Sectioned lung slides were mounted using DAPI and imaged using a Keyence All-in-one Fluorescence Microscope BZ-X series (Osaka, Japan).

### Statistics

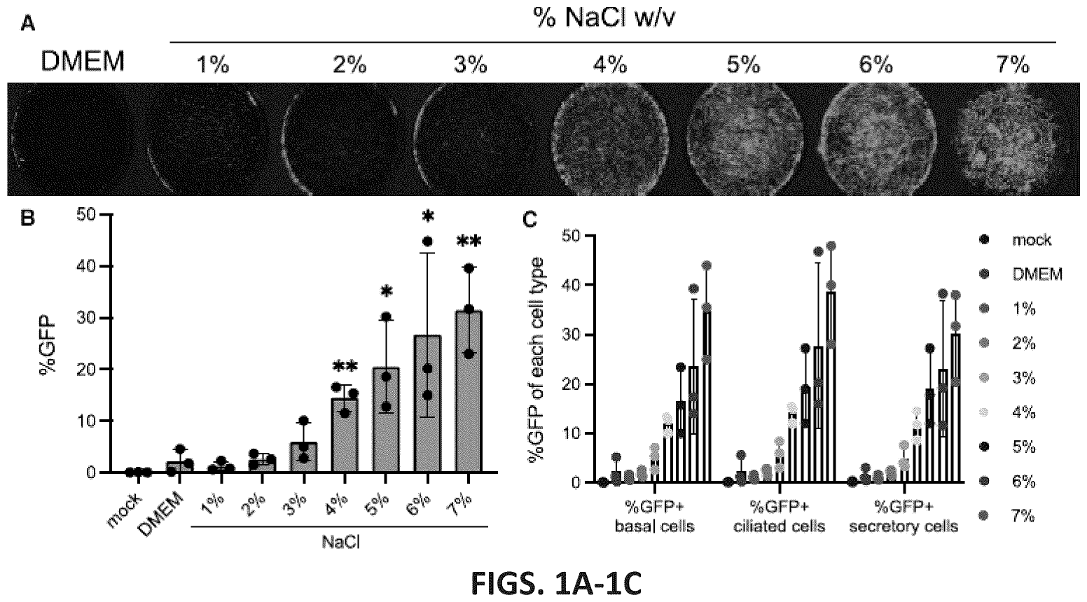

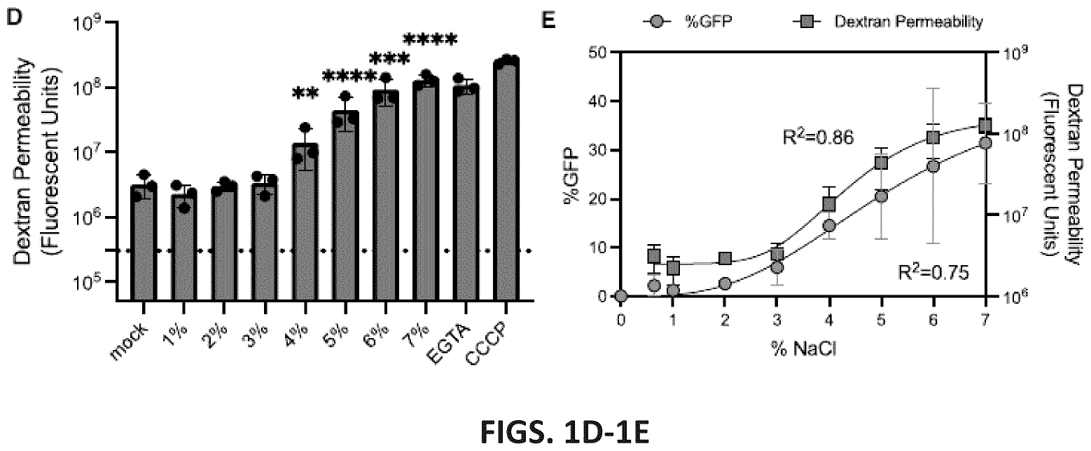

Student's two-tailed t-test, one-way analysis of variance (ANOVA) with Tukey's multiple comparison test, or two-way ANOVA with Dunnett's multiple comparison test were used to analyze differences in mean values between groups. Results are expressed as mean±SEM. P values≤0.05 were considered significant. R² values in FIG. 1E represent exponential growth of nonlinear regression.

### Results

Not All Vehicles that Decrease Transepithelial Resistance Enhance Gene Transfer.

Disrupting the epithelial tight junctions allows Ad5-based viral vectors access to their basolaterally localized coxsackie and adenovirus receptor (CXADR) (12). Unless otherwise specified, these studies use the Ad5 serotype. Several compounds were screened for their ability to reduce the transepithelial electrical resistance (TEER) of HAE and confer improved transduction of an Ad vector. First, reagents were applied to the apical surface of HAE for 5 minutes and then TEER was measured using an ohmmeter. Consistent with previous reports, lysophosphatidylcholine (LPC) (0.1%) and EGTA (250 mM) reduced TEER and increased gene transfer (2, 6). The clinically approved reagents doxorubicin (5 μM), NaCl (3.6%), and surfactant (Infasurf, 35 mg/ml) all decreased TEER (data not shown). In separate cultures, Ad-GFP (MOI=250) in 50 μl of DMEM combined 1:1 with the test reagents was applied to the apical surface for 2 hours. 5 days post-treatment, cells were imaged by fluorescence microscopy. Doxorubicin, surfactant, and 0.9% NaCl (PBS) did not enhance Ad gene transfer. Of note, the 3.6% NaCl formulation (mixed 1:1 from 7.2%) conferred remarkable transduction (data not shown). Based on this initial observation, this concentration of NaCl was selected for the many of the described Ad-based studies.

### Increasing Saline Tonicity Enhances Gene Transfer in a Dose and Time Dependent Manner.

To determine if increasing saline tonicity further improves Ad transduction in HAE, a dose response 1-7% NaCl formulation (final concentration; Table 1) with Ad-GFP (MOI=250). A dose dependent increase in transduction efficiency was observed with increasing NaCl tonicity (FIG. 1A, B). Basal, secretory, and ciliated cells were all transduced in a dose dependent manner (FIG. 1C,). HAE permeability to dextran also increased in the presence of 4-7% NaCl (FIG. 1D), which correlated with increasing GFP expression (FIG. 1E). The highest concentrations of NaCl (5-7%) cell viability as determined by flow cytometry (data not shown) and increased LDH release (data not shown). To visualize that cell surface integrity and ciliation remained intact, scanning electron microscopy (SEM) was performed on HAE that were untreated or treated with NaCl (3.6%) for 2 hours and observed no overt physical differences compared to the no NaCl control (data not shown). The therapeutic range of NaCl delivered to patients by aerosolization is up to 7% (21); however, based on the cell viability results, the delivered concentration of NaCl was restricted to 3.5-4.5% for subsequent in vitro studies (which lies within the clinically relevant therapeutic range (3-7%)).

TABLE 1
Molarity and osmolarity of NaCl, KCl, mannitol and NMDG gluconate up to 7%
Molarity and osmolarity of NaCl up to 7%. Tonicity, molarity, and osmolarity of
DMEM and 1-7% NaCl. Hypertonic indicated a NaCl solution >0.9% DMEM has additional
inorganic salts (i.e., CaCl₂, MgSO₄, KCl, and NaHCO₃) and is considered isotonic.

| | Isotonic | | | | | | | |
|---|---|---|---|---|---|---|---|---|
| Tonicity | DMEM | Hypertonic | | | | | | |
| % NaCl | 0.64% | 1% | 2% | 3% | 4% | 5% | 6% | 7% |
| Molarity | 110 mM | 170 mM | 340 mM | 510 mM | 680 mM | 860 mM | 1.03 mM | 1.2 M |
| Osmolarity | 219 | 342 | 684 | 1027 | 1369 | 1711 | 2053 | 2693 |
| | mOsmol/l | mOsmol/l | mOsmol/l | mOsmol/l | mOsmol/l | mOsmol/l | mOsmol/l | mOsmol/l |
| % KCl | 0.4% | 1% | 2% | 3% | 4% | 5% | 6% | 7% |
| Molarity | 50 mM | 130 mM | 270 mM | 400 mM | 540 mM | 670 mM | 800 mM | 940 mM |
| Osmolarity | 107 | 268 | 537 | 805 | 1073 | 1341 | 1610 | 1878 |
| | mOsmol/l | mOsmol/l | mOsmol/l | mOsmol/l | mOsmol/l | mOsmol/l | mOsmol/l | mOsmol/l |
| % mannitol | | 1% | 2% | 3% | 4% | 5% | 6% | 7% |
| Molarity | | 55 mM | 110 mM | 170 mM | 220 mM | 270 mM | 330 mM | 380 mM |
| Osmolarity | | 55 | 110 | 170 | 220 | 270 | 330 | 380 |
| | | mOsmol/l | mOsmol/l | mOsmol/l | mOsmol/l | mOsmol/l | mOsmol/l | mOsmol/l |
| % NMDG | | 1% | 2% | 3% | 4% | 5% | 6% | 7% |
| gluconate | | | | | | | | |
| Molarity | | 50 mM | 100 mM | 150 mM | 200 mM | 260 mM | 310 mM | 360 mM |
| Osmolarity | | 342 | 684 | 1027 | 1369 | 1711 | 2053 | 3693 |
| | | mOsmol/l | mOsmol/l | mOsmol/l | mOsmol/l | mOsmol/l | mOsmol/l | mOsmol/l |

Tonicity, molarity, and osmolarity of DMEM and 1-7% NaCl, KCl, mannitol, and N-methyl-n-glucamine (NMDG) gluconate. Hypertonic indicated a NaCl solution >0.9%. DMEM has additional inorganic salts (i.e., CaCl₂, MgSO₄, KCl, and NaHCO₃) and is considered isotonic.

It was next asked whether the length of exposure to NaCl or Ad impacted transduction in cultured HAE. As depicted schematically (data not shown), HAE were incubated with constant Ad-GFP for 120 min and varied times for NaCl (3.6%) at 5, 15, 30, 60, or 120 min. In parallel, HAE were incubated with constant NaCl (3.6%) and varied Ad-GFP incubation times. GFP expression was visualized 5 days post-transduction and quantified by flow cytometry (data not shown). The % GFP+cells increased incrementally with increasing temporal exposure to either Ad-GFP or NaCl, with significant increase at 1-hour Ad-GFP with constant NaCl (3.6%) exposure and maximal expression after a 2-hour co-treatment. This result established a standard 2-hour co-delivery protocol.

### NaCl-Mediated Ad Transduction Restores Anion Transport.

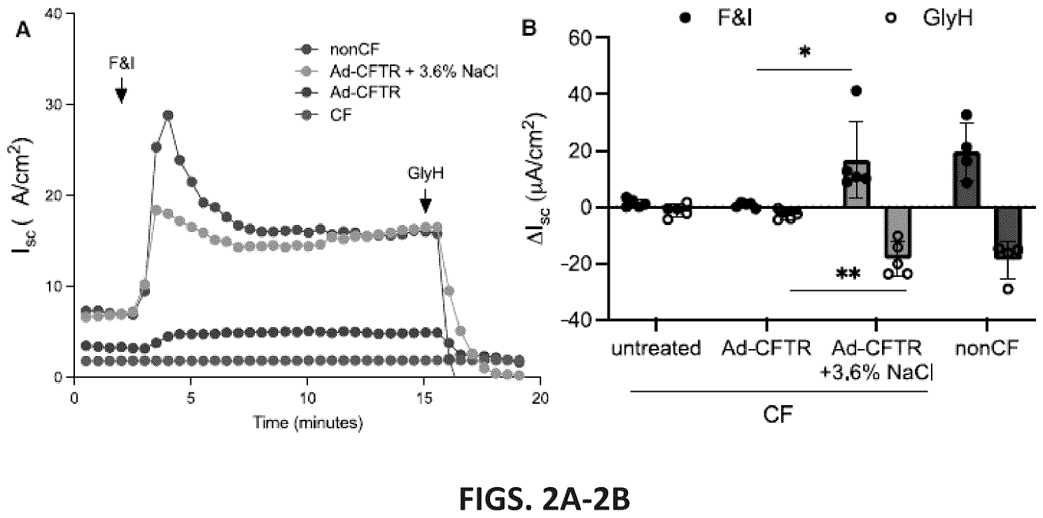

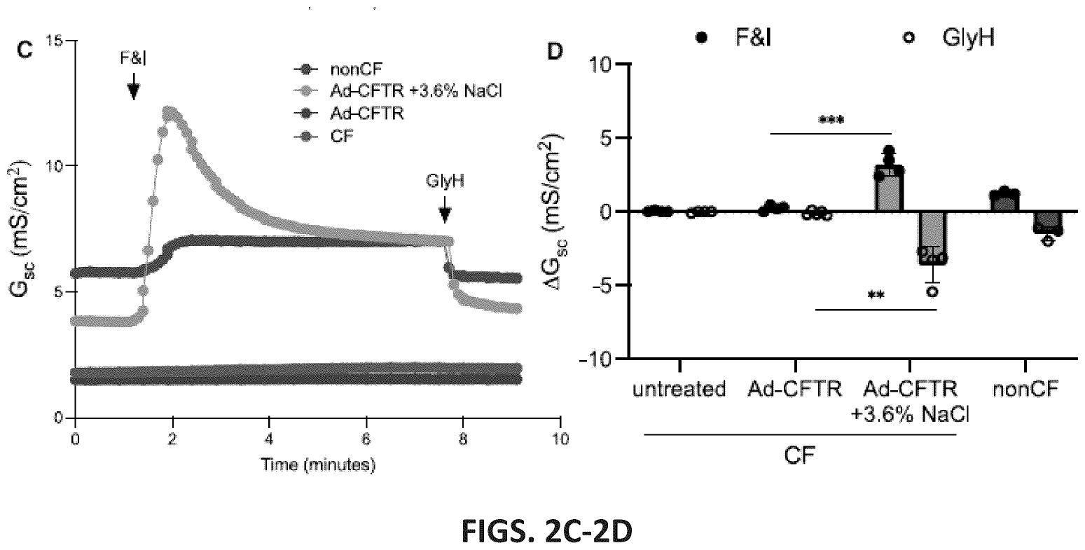

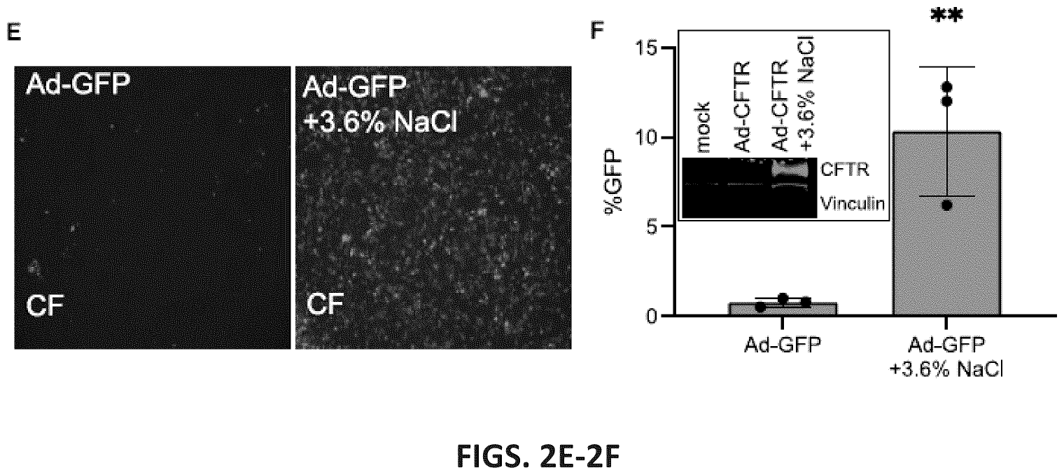

It was hypothesized that the NaCl conferred increase in transduction efficiency would enhance Ad-CFTR complementation of anion transport in CF HAE. Ad-CFTR formulated with NaCl (3.6%) was delivered to the apical surface of primary CF HAE for 2 hours. 5 days post-delivery, bioelectric properties were measured in Ussing chambers using the following protocol: amiloride, DIDS, forskolin and IBMX (F&I), and GlyH-101 (GlyH). Without NaCl (3.6%) formulation, no change in Cl-conductance was observed in response to the CFTR agonists forskolin and IBMX (F&I) or CFTR inhibitor GlyH in cultured primary CF epithelial treated with Ad-CFTR. However, cells that received Ad-CFTR and NaCl (3.6%) showed wild-type levels of Cl⁻ secretion as measured by short circuit current (FIG. 2A, B) and conductance (FIG. 2C, D). Parallel CF airway cultures were transduced as indicated to determine transgene expression by microscopy (FIG. 2F) or western blot (FIG. 2F, inset), respectively. These results suggest that NaCl formulation of Ad-CFTR improved gene transfer and conferred wild-type levels of CFTR mediated anion transport in CF HAE.

### Impact of Osmolarity, pH, and Entry on NaCl Enhanced Gene Transfer.

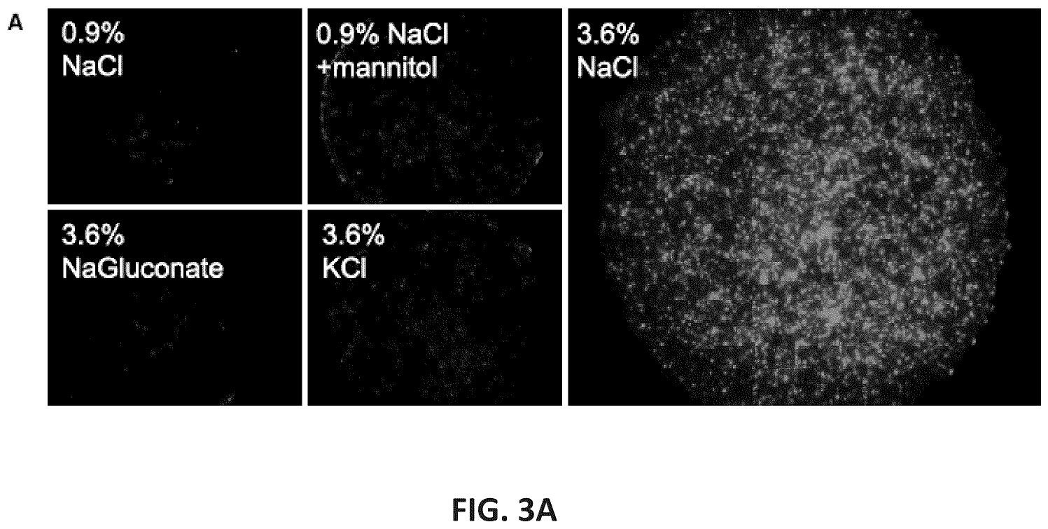

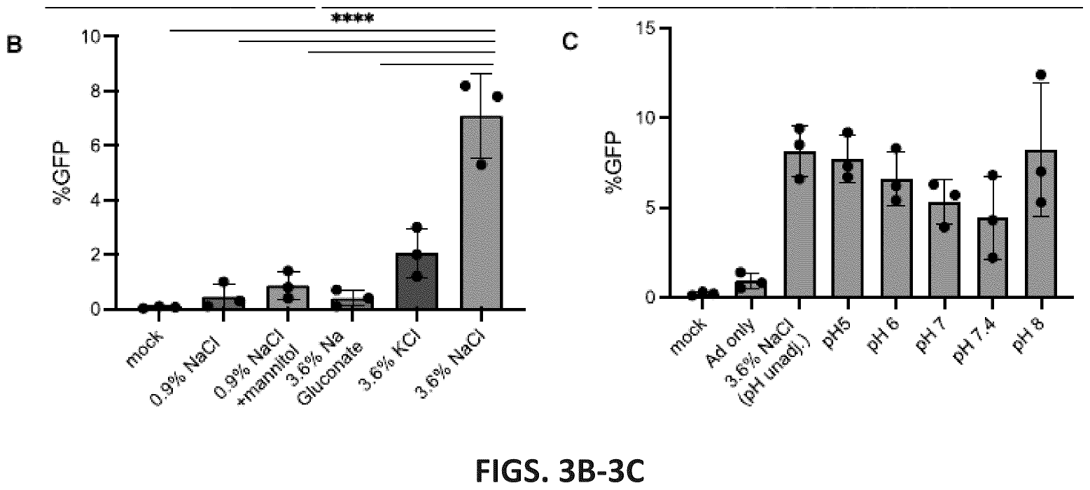

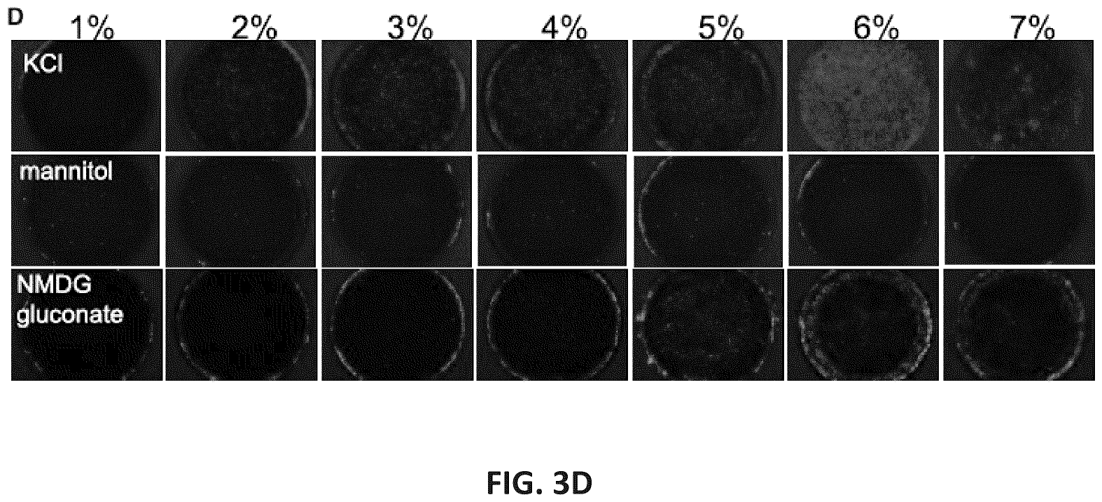

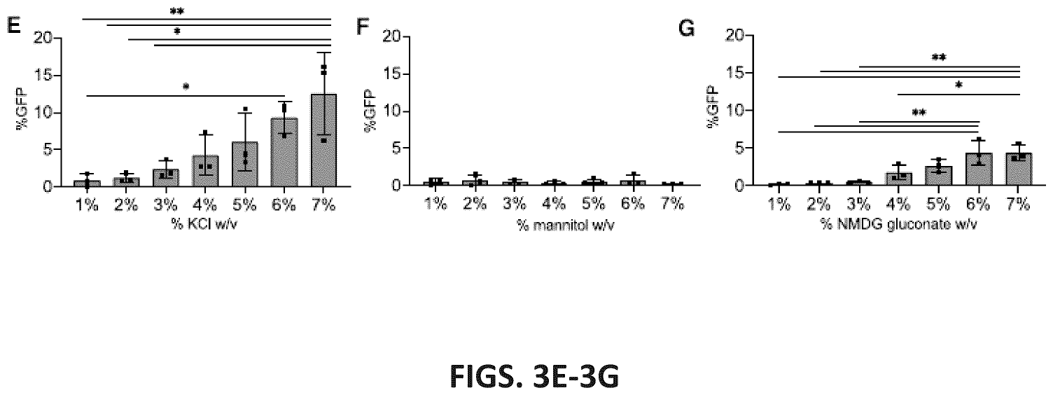

To investigate the mechanism for the enhanced transduction by NaCl formulations, 3.6% NaCl was compared to the ionic osmolytes 3.6% NaC₆H₁₁O₇ (NaGluconate), 3.6% KCl, 0.9% NaCl and 0.9% NaCl with the non-ionic osmolyte mannitol of equivalent osmolarity to the NaCl (3.6%) formulation (Table 2). The indicated regents were co-delivered with Ad-GFP to HAE for 2 h. Microscopy and flow cytometry quantification was performed 5 days later. NaCl (3.6%) conferred the greatest increase in transduction efficiency (FIG. 3A, B), whereas equivalent tonicity (3.6%) or osmolarity (620 mM, 1230 mOsmo/l) with ionic or non-ionic osmolytes did not significantly enhance gene transfer, indicating that equivalent osmolyte tonicity or osmolarity to NaCl (3.6%) was insufficient to enhance gene transfer. The unadjusted pH of 3.6% NaCl in deionized water is ˜6. To determine if the pH of NaCl formulation impacts transduction, the pH of the NaCl (3.6%) solution was adjusted to 5, 6, 7, 7.4, and 8. Using the standard Ad-GFP delivery and flow cytometry quantification protocols in HAE, no significant differences in % GFP+cells were observed across this pH range (FIG. 3C). To investigate another potential mechanism, it was determined if ionic strength was responsible for enhanced vector transduction. KCl (lower ionic strength than NaCl for a given percent solution), mannitol (uncharged sugar) and N-methyl-d-glucamine gluconate (charged sugar) at 1-7% tonicities were comparted (Table 1 and FIG. 3D). Dose-dependent increases were observed with KCl and N-methyl-d-glucamine gluconate whereas mannitol did not enhance gene transfer (FIG. 3E-G). The formulation was not dependent on osmolarity or pH, but ionic strength increased the Ad transduction efficiency.

TABLE 2
Molarity and osmolarity of ionic and non-ionic osmolytes

| | 3.6% NaCl | | 0.9 % NaCl | | 0.9% NaCl + mannitol | | 3.6% NaGluconate | | 3.6% KCl | |
|---|---|---|---|---|---|---|---|---|---|---|
| Molecular weight | 58.44 | g/mol | 58.44 | g/mol | Mannitol: 182.172 | g/mol | 590 | g/mol | 74.55 | g/mol |
| Tonicity (%) | 3.6% | | 0.9% | | 16% mannitol | | 3.6% | | 3.6% | |
| Molarity | 620 | mM | 150 | mM | 620 | mM | 61 | mM | 480 | mM |
| Osmolarity | 1230 | mOsmol/l | 310 | mOsmol/l | 1 230 | mOsmol/l | 120 | mOsmol/l | 970 | mOsmol/l |

Molecular weight, tonicity, molarity and osmolarity for NaCl (0.9% and 3.696, mannitol, NaGluconate and KCl are presented.
Table 2.
Molarity and osmolarity of ionic and non-ionic osmolytes.
Molecular weight, tonicity, molarity and osmolarity for NaCl (0.9% and 3.6%), mannitol, NaGluconate, and KCl are presented.

### NaCl-Mediated Gene Transfer Remains Receptor Dependent And Requires A Low pH Endosome

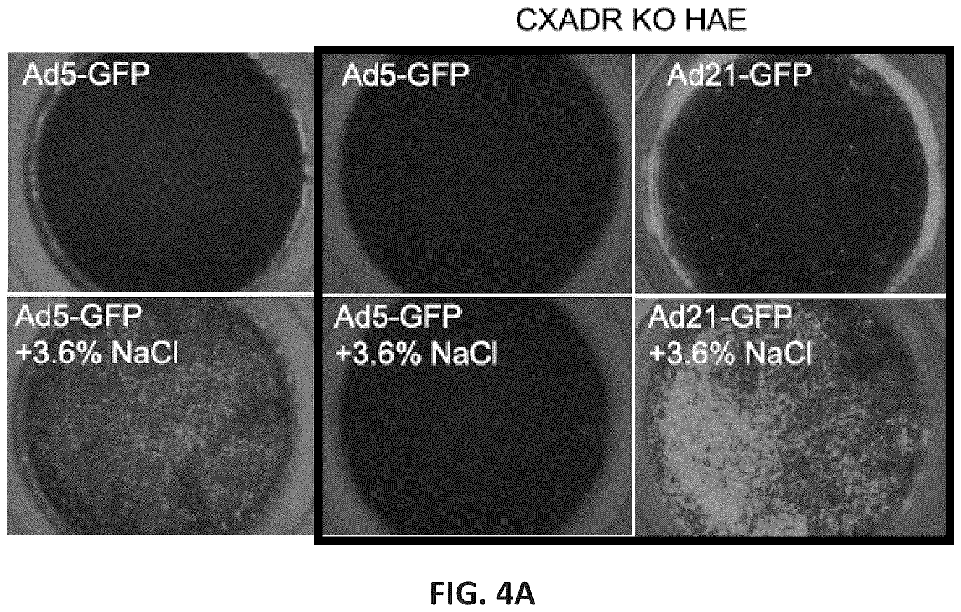

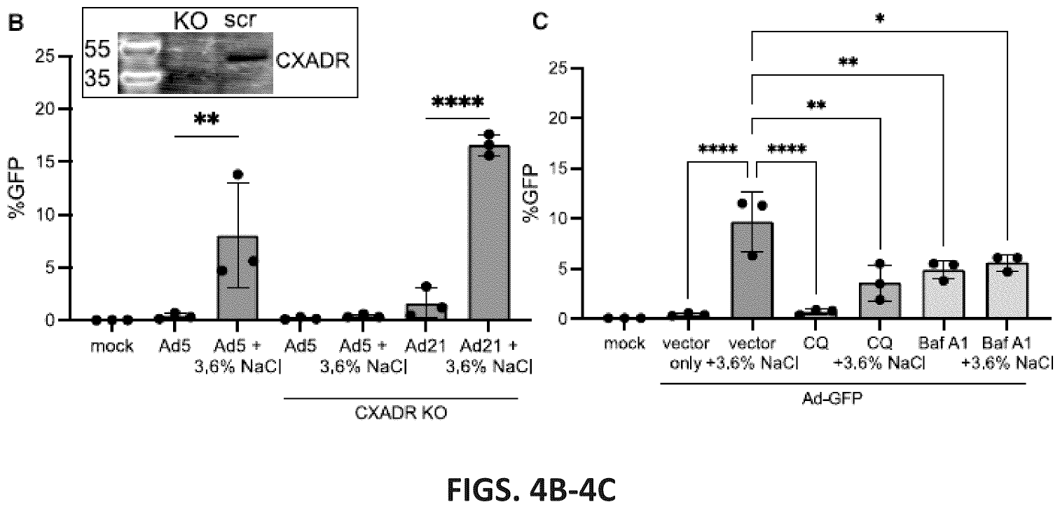

To investigate whether the NaCl enhanced Ad5 transduction is receptor dependent, CRISPR/Cas9 RNPs was used to knockout (KO) the Ad5 receptor Coxsackie and Adenovirus Receptor (CXADR) in primary human airway basal cells using electroporation (13, 14). The electroporated primary cells were seeded on transwell membranes and cultured at an air liquid interface until well-differentiated (˜21 days). CXADR^{KO} cells and donor-matched airway epithelia (HAE) were transduced with Ad5-GFP and Ad21-GFP in the presence or absence of NaCl (3.6%). Ad21 enters airway cells through a CXADR-independent mechanism using CD46 as a cellular receptor (15). Ad5+NaCl did not transduce CXADR^{KO} epithelia, whereas Ad21+NaCl readily transduced CXADR^{KO} cells (FIG. 4A, B). Following receptor engagement and internalization, Ad5 transduction requires escape from low pH endosomes. Inhibiting endosomal acidification with chloroquine (CQ) or bafilomycin A1 (Baf A1) significantly decreased the NaCl enhanced transduction (FIG. 4C). These results indicate NaCl enhanced transduction requires receptor engagement and low pH endosomal release.

It was next asked if this observation was specific to Ad5 or if a completely unrelated virus with a known receptor would also enhance transduction. Given the previous experience with measles virus (MeV) infecting airway epithelia (16, 17), the impact of formulating NaCl (4%) with wild-type MeV and delivering the virus to the apical surface was tested. The MeV receptor Nectin-4 is localized to the basolateral surface of human airway epithelial cells and MeV entry is known to occur via Nectin-4 at the basolateral membrane (17-19). Similar to Ad5, MeV infection following apical application of the virus occurred only in the presence of NaCl (4%). MeV with mutations in H protein that prevent the use of Nectin-4 as a receptor, termed Nectin-blind MeV (20), failed to infect HAE with or without NaCl (4%) formulation. These results indicate two potential mechanisms for enhanced entry including receptor dependence and, at least for Ad5, a low pH endosome step.

### NaCl Formulations Enhance Lentiviral and AAV Gene Transfer.

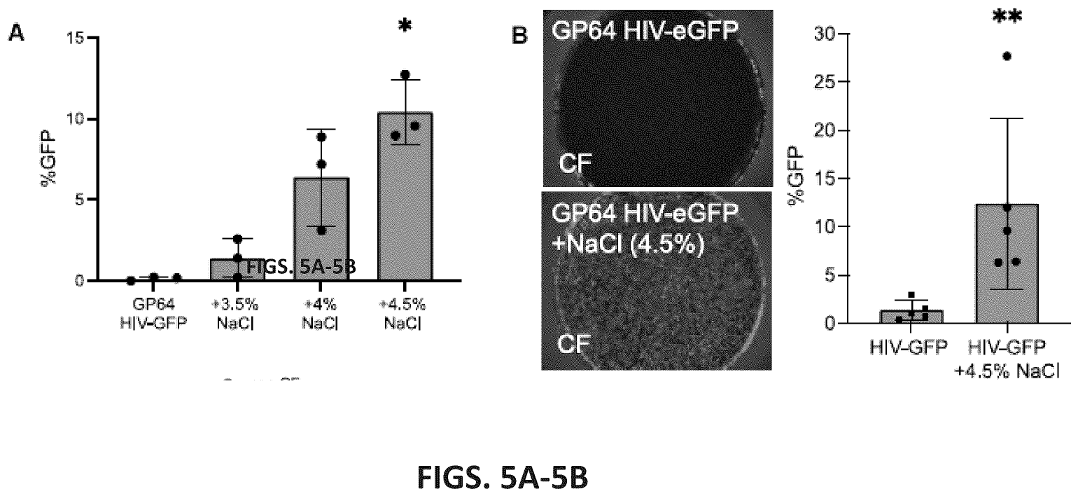

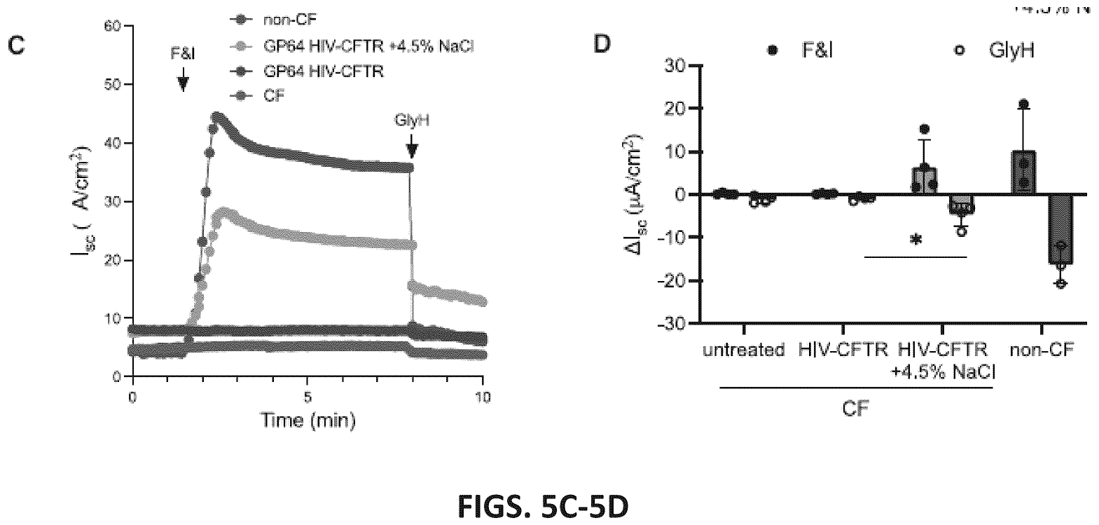

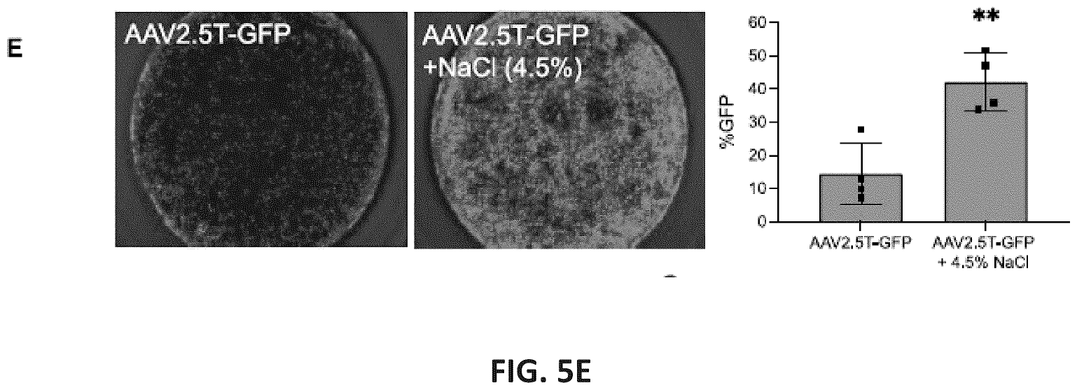

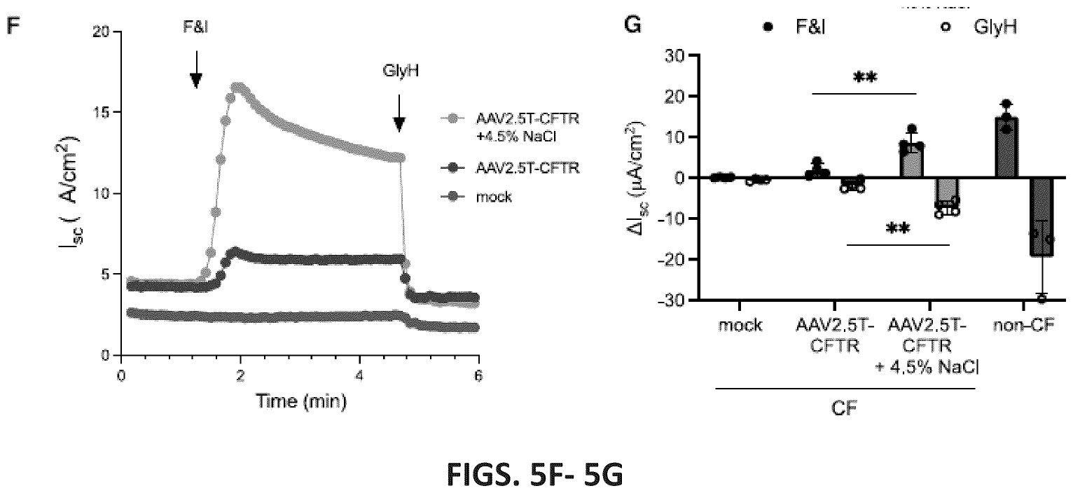

It was next asked if formulating lentiviral or adeno-associated viral (AAV) vectors with increasing concentrations of NaCl enhanced transduction. From the Ad-based studies, it was learned that ≤3% NaCl had little benefit on transduction while 5% NaCl formulation was noted to have negative effects on HAE integrity in vitro. Therefore, the experimental design was focused on a range of 3.5-4.5% NaCl. An HIV-based viral vector pseudotyped with the baculoviral GP64 envelope glycoprotein was applied to the apical surface of HAE with 3.5, 4 or 4.5% NaCl. A dose-dependent increase in GFP expression was observed in non-CF HAE (FIG. 5A) and 4.5% NaCl was then selected for lentiviral and AAV studies. Primary human CF cultures that received GP64 HIV-GFP (MOI=50) also showed remarkable levels of transduction (FIG. 5B). Notably, in the presence of NaCl (4.5%), GP64 HIV-CFTR restored the CFTR-dependent anion transport in primary CF HAE. Without NaCl formulation, improvements in CFTR-dependent currents were not observed (FIG. 5 C, D). Interestingly, NaCl-mediated entry did not enhance transduction of all pseudotyped lentiviral vectors. A screen of additional envelopes from Vesicular Stomatitis Virus (VSVG), Jaagsiekte Sheep Retrovirus (JSRV), and Baboon Endogenous Virus (BaEV) in the presence or absence of NaCl (4.5%) revealed that only GP64 and VSVG showed enhanced transduction (data not shown).

To test the effect of NaCl formulation on AAV-based vectors, the AAV2.5T capsid was selected based on previously established airway tropism (24). Similar to Ad and lentiviral studies, AAV-GFP (MOI=100,000 vg/cell) applied to the apical surface of HAE for 2 hours, which is reduced relative to the typical overnight treatments with AAV, with or without NaCl (4.5%). Doxorubicin (1 μM) was added to the basolateral media 24 h prior to the 2 h AAV application. A 2 h application was chosen to be consistent with the Ad and HIV protocols, however overnight applications of AAV are effective in the absence of NaCl. At the end of 2 hours, apical transductions were removed, and basolateral media was replaced with fresh media. Microscopy, flow cytometry quantification, and Ussing chamber assays were performed 2 weeks post-transduction. The epithelia treated with NaCl (4.5%) formulated AAV-GFP achieved significantly higher GFP expression (FIG. 5E) and increased anion channel activity responses to F&I and GlyH (FIG. 5F, G). These results confirm that transduction with NaCl formulation is enhanced across all three viral vector systems.

### Increased Saline Tonicity Enhances Gene Transfer Ex Vivo and In Vivo.

It was next asked if increased saline tonicity would enhance gene transfer in an ex vivo model. Using freshly excised newborn pig tracheal explants maintained on Surgifoam, Ad-GFP or AAVH22-GFP (a pig-tropic AA V capsid)±NaCl (5%) was applied to the apical surface by inverting the tissue in a culture dish for 2 hours. One week later, tissues were fixed and imaged by confocal microscopy. For both Ad and AAV, NaCl (5%) formulation resulted in improved gene transfer efficacy that was particularly striking using low power microscopy (data not shown). In the vector only condition, no appreciable expression was observed at low power, however expression was seen at higher magnifications (data not shown). Fluorescence was quantified by transduction area per high power field. Transduction with both Ad and AAVH22 was markedly enhanced in ex vivo tracheal explants when formulated with NaCl (5%) (data not shown).

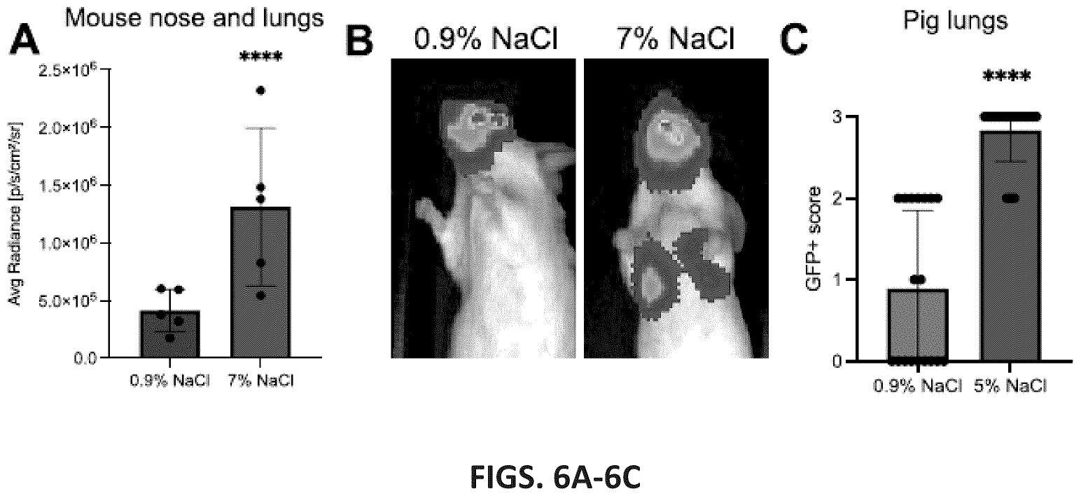

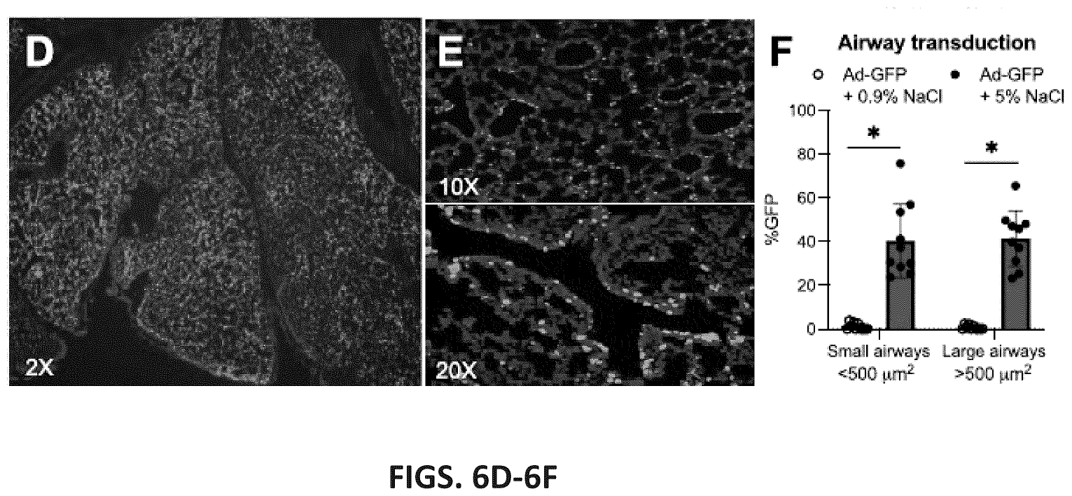

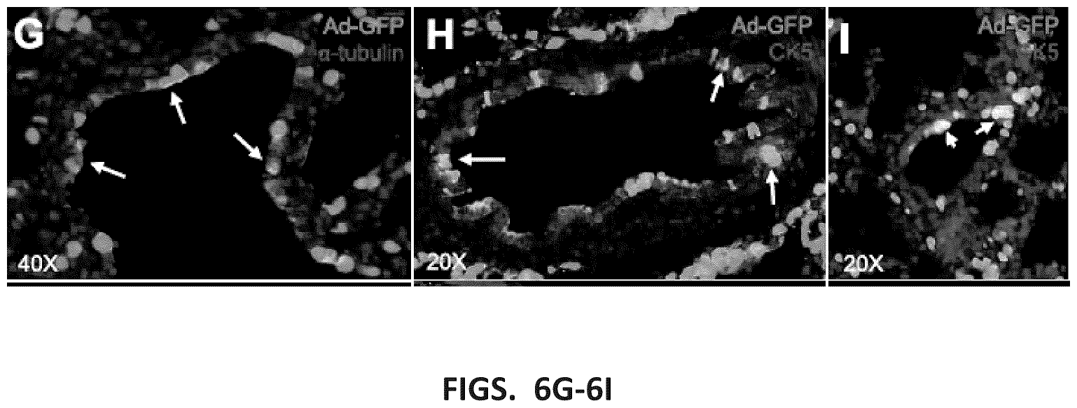

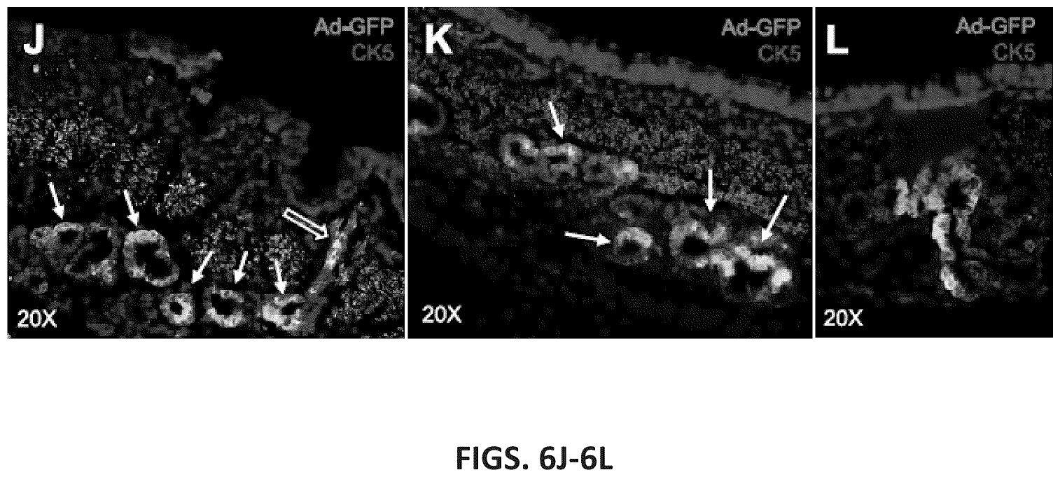

Improving viral transduction in vivo has broad implications for advancing gene therapy for lung, and other, diseases. It was first asked if vectors formulated with NaCl (7%) would enhance gene transfer to mouse airways/lungs. Ad-luciferase was formulated with NaCl (7%) or NaCl (0.9%) and delivered intratracheally. Five days post-delivery, quantification of bioluminescence showed that vectors formulated with 7% NaCl demonstrated significantly increased expression in the nose and lung relative to saline control mice (FIG. 6A, B). Nasal expression that exceeds lung expression following vector delivery via tracheal intubation is a reproducible phenomenon in mice.

To determine cell types transduced in vivo, a pig model that more closely recapitulates the anatomy and cell biology of human airways was used. Newborn pigs received Ad-GFP via intratracheal delivery with 0.9% NaCl or 5% NaCl. Five days after delivery, lungs were collected and GFP expression was visualized under a fluorescence dissecting microscope (data not shown). Lungs were divided into 20 regions for systematic quantification as previously described (data not shown) and scored based on level of expression (0=no expression, 1=low expression, 2=moderate expression, and 3=high expression) (data not shown). Formulation of Ad-GFP with NaCl (5%) resulted in a significantly increased the GFP+ score compared to 0.9% NaCl (FIG. 6C). The same assay was used to demonstrate that GP64 HIV-GFP formulated with NaCl (5%) significantly enhanced transduction over 0.9% NaCl (data not shown). GFP expression was widespread among airways of all sizes (FIG. 6D, E). Both large (>500 μm²) and small (<500 μm²) airways demonstrated significantly more airway transduction than Ad-GFP (0.9% NaCl) (FIG. 6F). Indeed, transduction of ciliated cells was observed (FIG. 6G) as well as cytokeratin 5 (CK5)+basal cells of the large (FIG. 6H) and small (FIG. 6I) airways. Additionally, high levels of transduction was observed in submucosal glands, including transduction of submucosal ductal cells (FIG. 6J-L). In summary, formulating either Ad or lentiviral vectors in 5-7% NaCl enhanced airway and alveolar gene transfer in mice and pigs.

### Discussion

The findings presented herein show that NaCl (3.6-7%) enhanced transduction efficiency of Ad, AAV, and lentivirus. This improved transduction restored functional CFTR anion transport in CF airway epithelia (HAE) across all three vector platforms. For adenovirus vectors, NaCl formulation enhanced transduction remains receptor dependent and requires a low pH endosomal escape. These in vitro findings of enhanced transduction also translated to two animal models where formulating Ad or lentiviral vectors with 5% or 7% NaCl increased transgene expression. Given that 3-7% NaCl is routinely aerosolized in people with CF, these findings are important implications for improving viral vector transduction for CF and other lung diseases. Formulating viral vectors with 3.6-7% NaCl can boost therapeutic efficacy, lower the therapeutic index, and overcome previous expression threshold limitations for pulmonary gene therapy.

Formulations including LPC (5), EGTA (6, 7), perfluorocarbon (25), and methylcellulose (8) have been looked at in in vitro and pre-clinical in vivo studies to enhance gene transfer. Interestingly, the discovery that Ad-based viral vectors use a basolateral receptor and gene transfer is boosted by tight junction disruption came to light after the first CF gene therapy clinical trials (4). Since none of the above-mentioned formulations are currently FDA approved for lung administration, each reagent would require a separate evaluation co-administration with a gene therapy vector. CF gene therapy clinical trials performed to date include vehicles with a 150 mM NaCl concentration, which is equivalent to 0.9% isotonic NaCl (reviewed in (4)). Viral vectors including AAV and lentiviral platforms are under investigation for CF gene therapy applications. 4D Molecular Therapeutics began a Phase I AAV clinical trial in 2022 (clinicaltrials.gov) and Spirovant and Carbon Biosciences are generating preclinical data to pursue clinical trials with AAV and bocavirus vectors. Historically, achieving sufficient gene transfer for phenotypic correction has been challenging. Efforts to over-come these challenges include directed evolution for capsid and envelope design, cassette modification including promoter, cDNA and polyadenylation signal, codon optimization, and immunomodulation. Using a formulation generally recognized as safe for use in people with CF such as NaCl (3-7%) can eliminate a hurdle for approval and advancement of a gene therapy reagent.

It is shown here that NaCl formulation enhances access to basal progenitor cells. Correcting sufficient stem cells by gene addition with integrating vectors such as lentivirus, piggyBac/Ad, or piggyBac/AAV would provide life-long correction for the patient. Alternatively, delivery of base editors, prime editors, or prime-editing-assisted site-specific integrase gene editing tools to stem cells using non-integrating vehicles such as Ad, AAV or LNPs would also provide life-long correction.

Effects of increased saline tonicity on viral vector transduction for gene therapy has not been previously investigated. Only one report exists demonstrating that increased saline tonicity does not enhance SARS-COV-2 viral entry (26). The lentiviral envelope screen supports that not all viral transduction is enhanced with increased saline tonicity It was observed that vectors with the GP64 and VSVG envelopes benefited from NaCl (4.5%) formulation, while the beta-retrovirus envelopes JSRV or BaEV did not. JSRV uses the GPI-linked apical receptor Hya12 while the BaEV uses SLC1A4 and SLClA5 as receptors. Although these receptors are present in HAE, transduction was not enhanced with NaCl formulation. These results suggest that entry pathways may influence the effectiveness of NaCl formulations. Indeed, different viruses have differing entry routes, including receptor-mediated endocytosis or fusion at the plasma membrane. Ad, AAV, GP64, and VSVG use receptor mediated endocytosis for entry and were enhanced by NaCl. The BeEV and JSRV confer membrane fusion at the cell surface for lentiviral entry and were not enhanced by NaCl.

It was originally suspected that the mechanism for NaCl-mediated enhanced transduction was due to the osmotic strength of the formulation. It is known that airway surface liquid volume is regulated in part by water movement through the cell. Osmotically driven water permeability is regulated by aquaporins to maintain airway epithelial cell homeostasis (27, 28). When the airway surface is hypertonic to epithelia, an electrochemical gradient generates a driving force for fluid movement across the epithelial barrier, causing hypertonic shock, followed by an adaptive regulatory increase in cell volume. The non-ionic osmolyte mannitol did not enhance transduction. Instead, it was learned that ionic strength is a key determinant in the mechanism by which NaCl formulation enhanced vector transduction. It is speculated that NaCl formulation may enhance transduction, in part, by increasing receptor access by disrupting tight junctions or by enhancing endosomal escape. It was observed that NaCl, KCl, and the charged sugar N-methyl-d-glucamine enhanced transduction in a dose-dependent manner, although NaCl had the greatest impact in a given percent solution. These findings suggest that Na⁺ regulation of the airway extracellular environment has the greatest impact on viral transduction.

As mentioned in the Results, a concentration of 3.6% final concentration of NaCl was serendipitously chosen for most of the studies using Ad because it was initially diluted 1:1 from a 7.2% pharmaceutical grade stock solution. For in vitro studies in HAE, the dose response results suggest that 4.5% NaCl may be the best compromise between efficacy and ultimate toxicity at doses of NaCl>6%. This was true for all vectors tested. Of note, attempts to improve gene transfer of cultured cells on plastic using hypertonic saline resulted in lifting of the cells. While all concentrations of NaCl were not tested in vivo, it was speculated that a dose-response would be seen. Using the in vitro results and common clinical dosages to choose the concentration of NaCl for the in vivo formulations. The results demonstrate that 5-7% NaCl formulations had a positive impact on gene transfer to the lungs of mice and pigs.

The goal of this study was to investigate whether FDA approved agents could improve lung gene therapy efficacy. Hypertonic saline is a routinely used therapy for people with CF and other lung conditions and could be rapidly adopted in vector formulations. Provided herein is evidence that formulating Ad, AAV or lentiviral vectors with NaCl can advance gene therapy development for at least CF. Importantly, this finding has broad implications for other genetic lung diseases such as alveolar type 2 deficiencies (ABCA3 Deficiency, Surfactant Protein B Deficiency) or Primary Ciliary Dyskinesia.

### BIBLIOGRAPHY

1. A. L. Cooney, et al., Lentiviral-mediated phenotypic correction of cystic fibrosis pigs. JCI Insight 1, (2016).
2. A. L. Cooney, et al., Widespread airway distribution and short-term phenotypic correction of cystic fibrosis pigs following aerosol delivery of piggyBac/adenovirus. Nucleic Acids Res 46, 9591-9600 (2018).
3. A. L. Cooney, et al., A Novel AAV-mediated Gene Delivery System Corrects CFTR Function in Pigs. Am J Respir Cell Mol Biol 61, 747-754 (2019).
4. A. L. Cooney, et al., Cystic Fibrosis Gene Therapy: Looking Back, Looking Forward. Genes (Basel) 9, (2018).
5. P. Cmielewski, et al., Lysophosphatidylcholine as an adjuvant for lentiviral vector mediated gene transfer to airway epithelium: effect of acyl chain length. Respir Res 11, 84 (2010).
6. G. Wang, et al., Increasing epithelial junction permeability enhances gene transfer to airway epithelia In vivo. Am J Respir Cell Mol Biol 22, 129-138 (2000).
7. G. Wang, et al., Influence of cell polarity on retrovirus-mediated gene transfer to differentiated human airway epithelia. J Virol 72, 9818-9826 (1998).
8. P. L. Sinn, et al., Viscoelastic gel formulations enhance airway epithelial gene transfer with viral vectors. Am J Respir Cell Mol Biol 32, 404-410 (2005).
9. L. N. Zhang, et al., Dual therapeutic utility of proteasome modulating agents for pharmaco-gene therapy of the cystic fibrosis airway. Mol Ther 10, 990-1002 (2004).
10. M. R. Elkins, P. T. Bye, Mechanisms and applications of hypertonic saline. J R Soc Med 104 Suppl 1, S2-5 (2011).
11. R. Tarran, et al., Rationale for hypertonic saline therapy for cystic fibrosis lung disease. Semin Respir Crit Care Med 28, 295-302 (2007).
12. K. J. Excoffon, et al., Isoform-specific regulation and localization of the coxsackie and adenovirus receptor in human airway epithelia. PLOS One 5, e9909 (2010).
13. E. Ortiz-Zapater, et al., Epithelial coxsackievirus adenovirus receptor promotes house dust mite-induced lung inflammation. Nat Commun 13, 6407 (2022).
14. S. Krishnamurthy, et al., Functional correction of CFTR mutations in human airway epithelial cells using adenine base editors. Nucleic Acids Res 49, 10558-10572 (2021).
15. D. Sirena, et al., The human membrane cofactor CD46 is a receptor for species B adenovirus serotype 3. J Virol 78, 4454-4462 (2004).
16. M. D. Muhlebach, et al., Adherens junction protein nectin-4 is the epithelial receptor for measles virus. Nature 480, 530-533 (2011).
17. B. K. Singh, et al., Cell-to-Cell Contact and Nectin-4 Govern Spread of Measles Virus from Primary Human Myeloid Cells to Primary Human Airway Epithelial Cells. J Virol 90, 6808-6817 (2016).
18. M. Mateo, et al., Different roles of the three loops forming the adhesive interface of nectin-4 in measles virus binding and cell entry, nectin-4 homodimerization, and heterodimerization with nectin-1. J Virol 88, 14161-14171 (2014).
19. B. K. Singh, et al., The Nectin-4/Afadin Protein Complex and Intercellular Membrane Pores Contribute to Rapid Spread of Measles Virus in Primary Human Airway Epithelia. J Virol 89, 7089-7096 (2015).
20. V. H. Leonard, et al., Measles virus blind to its epithelial cell receptor remains virulent in rhesus monkeys but cannot cross the airway epithelium and is not shed. J Clin Invest 118, 2448-2458 (2008).
21. P. L. Sinn, et al., Novel GP64 envelope variants for improved delivery to human airway epithelial cells. Gene Ther 24, 674-679 (2017).
22. P. L. Sinn, et al., Gene transfer to respiratory epithelia with lentivirus pseudotyped with Jaagsiekte sheep retrovirus envelope glycoprotein. Hum Gene Ther 16, 479-488 (2005).
23. A. Girard-Gagnepain, et al., Baboon envelope pseudotyped LVs outperform VSV-G-LVs for gene transfer into early-cytokine-stimulated and resting HSCs. Blood 124, 1221-1231 (2014).
24. K. J. Excoffon, et al., Directed evolution of adeno-associated virus to an infectious respiratory virus. Proc Natl Acad Sci U S A 106, 3865-3870 (2009).
25. J. A. Kazzaz, et al., Perfluorochemical liquid-adenovirus suspensions enhance gene delivery to the distal lung. Pulm Med 2011, 918036 (2011).
26. R. R. G. Machado, et al., Inhibition of Severe Acute Respiratory Syndrome Coronavirus 2 Replication by Hypertonic Saline Solution in Lung and Kidney Epithelial Cells. ACS Pharmacol Transl Sci 4, 1514-1527 (2021).
27. S. M. Kreda, et al., Expression and localization of epithelial aquaporins in the adult human lung. Am J Respir Cell Mol Biol 24, 224-234 (2001).
28. A. S. Verkman, M. A. Matthay, Y. Song, Aquaporin water channels and lung physiology. Am J Physiol Lung Cell Mol Physiol 278, L867-879 (2000).
29 S. Y. Graeber, et al., Hypertonic saline is effective in the prevention and treatment of mucus obstruction, but not airway inflammation, in mice with chronic obstructive lung disease. Am J Respir Cell Mol Biol 49, 410-417 (2013).
30. V. Havasi, et al., Inhibitory effects of hypertonic saline on P. aeruginosa motility. J Cyst Fibros 7, 267-269 (2008).
31. R. Bartoszewski, S. Matalon, J. F. Collawn, Ion channels of the lung and their role in disease pathogenesis. Am J Physiol Lung Cell Mol Physiol 313, L859-L872 (2017).
32. S. K. Rai, J. C. DeMartini, A. D. Miller, Retrovirus vectors bearing jaagsiekte sheep retrovirus Env transduce human cells by using a new receptor localized to chromosome 3p21.3. J Virol 74, 4698-4704 (2000).
33. P. H. Karp, et al., An in vitro model of differentiated human airway epithelia. Methods for establishing primary cultures. Methods Mol Biol 188, 115-137 (2002).
34. R. D. Anderson, et al., A simple method for the rapid generation of recombinant adenovirus vectors. Gene Ther 7, 1034-1038 (2000).
35. P. L. Sinn, et al., Lentiviral Vectors Pseudotyped with Filoviral Glycoproteins. Methods Mol Biol 1628, 65-78 (2017).
36. A. L. Cooney, et al., Lentiviral vectors transduce lung stem cells without disrupting plasticity. Mol Ther Nucleic Acids 25, 293-301 (2021).
37. Y. Wang, S. Bergelson, M. Feschenko, Determination of Lentiviral Infectious Titer by a Novel Droplet Digital PCR Method. Hum Gene Ther Methods 29, 96-103 (2018).

What is claimed is:

1. A method to enhance the delivery of one or more gene therapy vectors to cells comprising contacting said cells with a hypertonic solution before, after or simultaneously with said one or more gene therapy vectors as compared to a control.

2. A method to enhance the delivery of one or more gene therapy vectors to a subject in need thereof comprising administering to said subject a hypertonic solution before, after or simultaneously with said one or more gene therapy vectors as compared to a control.

3. A method to restore cystic fibrosis transmembrane conductance regulator (CFTR) function to cells of subjects with cystic fibrosis comprising contacting said cells with a hypertonic solution before, after or simultaneously with said one or more gene therapy vectors.

4. The method of claim 1, wherein the cells comprise epithelial cells.

5. The method of claim 1, wherein the cells are pulmonary epithelial cells.

6. The method of claim 2, wherein the subject as a disease or disorder of a hollow organ.

7. The method of claim 6, wherein the hollow organ comprises respiratory tract, lung, sinus, intestinal tract, mouth, esophagus, stomach, bladder, gallbladder, reproductive tract or heart.

8. The method of claim 6, wherein the disease or disorder is a genetic disease or disorder.

9. The method of claim 6, wherein the disease or disorder comprises cystic fibrosis, primary ciliary dyskinesia (PCD), surfactant deficiencies or underlying causes of interstitial lung disease, atopic lung disease, asthma, bronchiectasis, bronchiolitis obliterans, cystic lung diseases, Alpha-1 antitrypsin (AAT) deficiency, alveolar diseases in alveolar type 1 or 2 cells (e.g., alveolar type 2 deficiencies (ABCA3 Deficiency, Surfactant Protein B Deficiency)), lung cancer, Crohn's, ulcerative colitis, colon cancer, Lynch syndrome, familial adenomatous polyposis (FAP), inflammatory bowel disease, oral cancer, achalasia, GERD, esophageal cancer, Barrett's syndrome, stomach cancer, bladder cancer, bladder exstrophy, incontinence, Giltelman syndrome, gallstones, cardiomyopathies, channelopathies, heart disease, inherited heart conditions, hypertrophic cardiomyopathy (HCM), idiopathic or familial dilated cardiomyopathy (DCM), arrhythmogenic right ventricular cardiomyopathy (ARVC), restrictive cardiomyopathy, Carney complex, cancer, pancreatic diseases, kidney diseases, vascular diseases, bone diseases, spleen diseases, ear diseases, skin diseases, and/or blood diseases.

10. The method of claim 9, wherein the cancer comprises a sarcoma, carcinoma, lung cancer, adenocarcinoma, adenocarcinoma of the lung, squamous carcinoma, squamous carcinoma of the lung, malignant mixed mullerian tumor, head and/or neck cancer, breast cancer, esophageal cancer, mouth cancer, tongue cancer, gum cancer, skin cancer, muscle cancer, heart cancer, liver cancer, bronchial cancer, cartilage cancer, bone cancer, stomach cancer, prostate cancer, testicular cancer, ovarian cancer, cervical cancer, endometrial cancer, uterine cancer, pancreatic cancer, colon cancer, rectal cancer, colorectal, gastric cancer, kidney cancer, bladder cancer, spleen cancer, thymus cancer, thyroid cancer, brain cancer, neuronal cancer, mesothelioma, gall bladder cancer, ocular cancer, joint cancer, glioblastoma, neuroblastoma, hereditary non-polyposis cancer (HNPC), and/or colitis-associated cancer.

11. The method of claim 1, wherein the gene therapy vector is a viral vector.

12. The method of claim 11, wherein the viral vector comprises adenovirus, adeno-associated virus (AAV), lentivirus, retrovirus, bocavirus, oncolytic vector, and/or virus like particles (VLPs).

13. The method of claim 1, wherein the gene therapy vector is a plasmid, viral nucleic acid, RNA, peptide, protein, phage nucleic acid, phage, cosmid, artificial chromosome, or via transfer of genetic material in cells or carriers such as cationic liposomes or nanoparticles.

14. The method of claim 1, wherein the hypertonic solution is a hypertonic salt solution.

15. The method of claim 14, wherein the hypertonic salt solution comprises one or more monovalent salts.

16. The method of claim 15, wherein the one or more monovalent salts comprise sodium chloride (NaCl), potassium chloride (KCl), and/or lithium chloride (LiCl).

17. The method of claim 14, wherein the hypertonic salt solution comprises one or more divalent salts.

18. The method of claim 17, wherein the one or more divalent salts comprise calcium chloride (CaCl₂), magnesium sulfate (MgSO₄), magnesium chloride (MgCl₂), zinc cholirde (ZnCl₂), zinc sulfate (ZnSO₄), calcium lactate (C₆H₁₀CaO₆), ferrous sulfate (FeSO₄), calcium glycerylphosphate and/or ferrous chloride (FeCl₂) or charged sugar (e.g., N-methyl-D-gluconate) with ionic strength.

19. The method of claim 14, wherein the salt concentration is from about 1% to about 8%.

20. The method of claim 19, wherein the salt concentration is from about 3.5% to about 7%.

21. The method of claim 19, wherein the salt concentration is from about 3.5% to about 4.5% NaCl.

22. The method of 2, wherein the hypertonic solution is administered by inhalation, instillation, topical delivery, or injection.

23. The method of claim 22, wherein the injection is an intra-tumoral injection.
